SEQUENCE LISTING (119 sequences)

SEQ ID NO: 1 - protein, 216 aa, Mus musculus
DIQMTQSPSSLSASVGDRVTITCRASQSVSSAVAWYQQKPGKAPKLLIYSASSLYSGVPSRFSGSRSGTDFTLTISSLQPEDFATYYCQQYLAYQSPLTFGQGTKVEIKTRVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 227 aa, Mus musculus
EVQLVESGGGLVQPGGSLRLSCAASGFNVSYSSIHWVRQAPGKGLEWVASISSYYSYTSYADSVKGRFTISADTSKNTAYLQMNSLRAEDTAVYYCARYEEWAYYSSEMAFDYWGQGTLVTVSSASTKGPSVFPLAPSSKSTAGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 3 - protein, 6 aa, Artificial Sequence
  synthetic peptide
YGSSPI

SEQ ID NO: 4 - protein, 8 aa, Artificial Sequence
  synthetic peptide
YLAYQSPL

SEQ ID NO: 5 - protein, 6 aa, Artificial Sequence
  synthetic peptide
SSSSLI

SEQ ID NO: 6 - protein, 6 aa, Artificial Sequence
  synthetic peptide
SYHWLI

SEQ ID NO: 7 - protein, 6 aa, Artificial Sequence
  synthetic peptide
YPSLLI

SEQ ID NO: 8 - protein, 6 aa, Artificial Sequence
  synthetic peptide
FYSSYI

SEQ ID NO: 9 - protein, 6 aa, Artificial Sequence
  synthetic peptide
VSYSSI

SEQ ID NO: 10 - protein, 6 aa, Artificial Sequence
  synthetic peptide
FSSSSI

SEQ ID NO: 11 - protein, 10 aa, Artificial Sequence
  synthetic peptide
YISPSSGYTS

SEQ ID NO: 12 - protein, 10 aa, Artificial Sequence
  synthetic peptide
SISSYYSYTS

SEQ ID NO: 13 - protein, 10 aa, Artificial Sequence
  synthetic peptide
YIYSYSGSTS

SEQ ID NO: 14 - protein, 10 aa, Artificial Sequence
  synthetic peptide
SIYPSYGYTS

SEQ ID NO: 15 - protein, 10 aa, Artificial Sequence
  synthetic peptide
YISSYSGYTS

SEQ ID NO: 16 - protein, 9 aa, Artificial Sequence
  synthetid peptide
SIYYSGYTS

SEQ ID NO: 17 - protein, 15 aa, Artificial Sequence
  synthetic peptide
HPWYWTNYWFYEYGL

SEQ ID NO: 18 - protein, 13 aa, Artificial Sequence
  synthetic peptide
YEEWAYYSSEMAF

SEQ ID NO: 19 - protein, 15 aa, Artificial Sequence
  synthetic peptide
HDSWYPYYEQRQWGL

SEQ ID NO: 20 - protein, 13 aa, Artificial Sequence
  synthetic peptide
YQEQYGSYFGGAL

SEQ ID NO: 21 - protein, 12 aa, Artificial Sequence
  synthetic peptide
PAPQLGLGEKGL

SEQ ID NO: 22 - protein, 17 aa, Artificial Sequence
  synthetic peptide
YQHYYYGWGYRYLSSAM

SEQ ID NO: 23 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 24 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 25 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 26 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 27 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 28 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 29 - protein, 20 aa, Homo sapiens
KYLATASTMDHARHGFLPRH

SEQ ID NO: 30 - protein, 16 aa, Homo sapiens
ENPWHFFKNIVTPRTP

SEQ ID NO: 31 - protein, 19 aa, Homo sapiens
LSRFSWGAEGQRPGFGYGG

SEQ ID NO: 32 - protein, 25 aa, Homo sapiens
AQGTLSKIFKLGGRDSRSGSPMARR

SEQ ID NO: 33 - protein, 20 aa, Homo sapiens
GQFRVIGPRHPIRALVGDEV

SEQ ID NO: 34 - protein, 20 aa, Homo sapiens
MEVGWYRPPFSRWHLYRNGK

SEQ ID NO: 35 - protein, 16 aa, Homo sapiens
HCLGKWLGHPDKFVGI

SEQ ID NO: 36 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 37 - protein, 529 aa, Homo sapiens [sequence omitted]

SEQ ID NO: 38 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 39 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 40 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 41 - protein, 1247 aa, Homo sapiens
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 42 - protein, 33 aa, Homo sapiens
LQLQPFPQPQLPYPQPQLPYPQPQLPYPQPQPF

SEQ ID NO: 43 - protein, 33 aa, Homo sapiens
LQLQPFPQPELPYPQPELPYPQPELPYPQPQPF

SEQ ID NO: 44 - protein, 18 aa, Homo sapiens
QQYPSGQGSFQPSQQNPQ

SEQ ID NO: 45 - protein, 12 aa, Homo sapiens
QPFPQPEQPFPW

SEQ ID NO: 46 - protein, 25 aa, Artificial Sequence
  synthetic peptide
MPALWLGCCLCFSLLLPAARNLAGT

SEQ ID NO: 47 - DNA, 20 bp, Artificial Sequence
  synthetic oligonucleotide
cgcaacttattactgtcagc

SEQ ID NO: 48 - DNA, 19 bp, Artificial Sequence
  synthetic oligonucleotide
agacggtgaccagggttcc

SEQ ID NO: 49 - protein, 6 aa, Artificial Sequence
  synthetic peptide
VSSAVA

SEQ ID NO: 50 - protein, 6 aa, Artificial Sequence
  synthetic peptide
SASSLY

SEQ ID NO: 51 - protein, 6 aa, Artificial Sequence
  Synthetic peptide
SYWYPV

SEQ ID NO: 52 - protein, 7 aa, Artificial Sequence
  Synthetic peptide
SPWWGPI

SEQ ID NO: 53 - protein, 6 aa, Artificial Sequence
  Synthetic peptide
SSSSLI

SEQ ID NO: 54 - protein, 8 aa, Artificial Sequence
  synthetic peptide
YVRYYGPI

SEQ ID NO: 55 - protein, 6 aa, Artificial Sequence
  synthetic peptide
LSSSSI

SEQ ID NO: 56 - protein, 6 aa, Artificial Sequence
  synthetic peptide
FSYYSI

SEQ ID NO: 57 - protein, 6 aa, Artificial Sequence
  synthetic peptide
VYYSSI

SEQ ID NO: 58 - protein, 6 aa, Artificial Sequence
  synthetic peptide
ISSSSI

SEQ ID NO: 59 - protein, 10 aa, Artificial Sequence
  synthetic peptide
SISSYYGYTY

SEQ ID NO: 60 - protein, 10 aa, Artificial Sequence
  synthetic peptide
SIYPYSGYTS

SEQ ID NO: 61 - protein, 10 aa, Artificial Sequence
  synthetic peptide
SISPSSSYTS

SEQ ID NO: 62 - protein, 10 aa, Artificial Sequence
  synthetic peptide
SISPSYGSTY

SEQ ID NO: 63 - protein, 17 aa, Artificial Sequence
  synthetic peptide
NDDWYIWDWYYTRWYGL

SEQ ID NO: 64 - protein, 18 aa, Artificial Sequence
  synthetic peptide
YSYEYWRLYLFQYFWLGL

SEQ ID NO: 65 - protein, 15 aa, Artificial Sequence
  synthetic peptide
WYWYDYFWWWHQEAL

SEQ ID NO: 66 - protein, 17 aa, Artificial Sequence
  synthetic peptide
YWHWWGFSYWAYGYYGF

SEQ ID NO: 67 - protein, 6 aa, Artificial Sequence
  synthetic peptide
  MISC_FEATURE (1)..(1): X = L or V
  MISC_FEATURE (3)..(3): X = S or Y
XSXSSI

SEQ ID NO: 68 - protein, 10 aa, Artificial Sequence
  synthetic peptide
  MISC_FEATURE (7)..(7): X = G or S
  MISC_FEATURE (10)..(10): X = Y or S
SISSYYXYTX

SEQ ID NO: 69 - protein, 62 aa, Artificial Sequence
  synthetic peptide
GQFRVIGPRHPIRALVGDEVELPCRISPGKNATGMEVGWYRPPFSRVVHLYRNGKDQDGDQA

SEQ ID NO: 70 - protein, 63 aa, Artificial Sequence
  synthetic peptide
SHGRTQDENPVVHFFKNIVTPRTPPPSQGKGRGLSLSRFSWGAEGQRPGFGYGGRASDYKSCG

SEQ ID NO: 71 - protein, 52 aa, Artificial Sequence
  synthetic peptide
GCASQKRPSQRHGSKYLATASTMDHARHGFLPRHRDTGILDSIGRFFGGDRG

SEQ ID NO: 72 - protein, 42 aa, Artificial Sequence
  synthetic peptide
ASDYKSAHKGFKGVDAQGTLSKIFKLGGRDSRSGSPMARRCG

SEQ ID NO: 73 - protein, 33 aa, Artificial Sequence
  synthetic peptide
SHGRTQDENPVVHFFKNIVTPRTPPPSQGKGCG

SEQ ID NO: 74 - protein, 37 aa, Artificial Sequence
  synthetic peptide
SQGKGRGLSLSRFSWGAEGQRPGFGYGGRASDYKSCG

SEQ ID NO: 75 - protein, 27 aa, Artificial Sequence
  synthetic peptide
GQFRVIGPRHPIRALVGDEVELPCRIS

SEQ ID NO: 76 - protein, 45 aa, Artificial Sequence
  synthetic peptide
DEVELPCRISPGKNATGMEVGWYRPPFSRVVHLYRNGKDQDGDQA

SEQ ID NO: 77 - protein, 7 aa, Artificial Sequence
  synthetic peptide
PQPELPY

SEQ ID NO: 78 - protein, 17 aa, Artificial Sequence
  synthetic peptide
LQLQPFPQPQLPYPQPE

SEQ ID NO: 79 - protein, 17 aa, Artificial Sequence
  synthetic peptide
LQLQPFPQPQLPYPQPQ

SEQ ID NO: 80 - protein, 980 aa, Homo sapiens
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPADAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 81 - protein, 65 aa, Homo sapiens
AYQAEPNTCATAQGEGNIKKNRHPDFLPYDHARIKLKVESSPSRSDYINASPIIEHDPRMPAYIA

SEQ ID NO: 82 - protein, 35 aa, Homo sapiens
GPLSHTIADFWQMVWESGCTVIVMLTPLVEDGVKQ

SEQ ID NO: 83 - protein, 56 aa, Homo sapiens
GASLYHVYEVNLVSEHIWCEDFLVRSFYLKNVQTQETRTLTQFHFLSWPAEGTPAS

SEQ ID NO: 84 - protein, 37 aa, Homo sapiens
EHVRDQRPGLVRSKDQFEFALTAVAEEVNAILKALPQ

SEQ ID NO: 85 - protein, 46 aa, Homo sapiens
TCATAQGEGNIKKNRHPDFLPYDHARIKLKVESSPSRSDYINASPI

SEQ ID NO: 86 - protein, 29 aa, Homo sapiens
KNRHPDFLPYDHARIKLKVESSPSRSDYI

SEQ ID NO: 87 - protein, 26 aa, Homo sapiens
VSEHIWCEDFLVRSFYLKNVQTQETR

SEQ ID NO: 88 - protein, 25 aa, Homo sapiens
LVRSFYLKNVQTQETRTLTQFHFLS

SEQ ID NO: 89 - protein, 37 aa, Homo sapiens; identical to SEQ ID NO: 84

SEQ ID NO: 90 - protein, 9 aa, Homo sapiens
ALTAVAEEV

SEQ ID NO: 91 - protein, 79 aa, Homo sapiens
FVNQHLCGSHLVEALYLVCGERGFFYTPKTRREAEDLQVGQGAGSLQPLALEGSLQKRGIVEQCCTSICSLYQLENYCN

SEQ ID NO: 92 - protein, 62 aa, Homo sapiens
SHLVEALYLVCGERGFFYTPKTRREAEDLQVGQVELGGGPGAGSLQPLALEGSLQKRGIVEQ

SEQ ID NO: 93 - protein, 28 aa, Homo sapiens
SHLVEALYLVCGERGFFYTPKTRREAED

SEQ ID NO: 94 - protein, 28 aa, Homo sapiens
GGGPGAGSLQPLALEGSLQKRGIVEQCG

SEQ ID NO: 95 - protein, 51 aa, Homo sapiens
GERGFFYTPKTRREAEDLQVGQVELGGGPGAGSLQPLALEGSLQKRGIVEQ

SEQ ID NO: 96 - protein, 31 aa, Homo sapiens
GERGFFYTPKTRREAEDLQVGQVELGGGPGA

SEQ ID NO: 97 - protein, 13 aa, Homo sapiens
YGYTHLSTGDLLR

SEQ ID NO: 98 - protein, 16 aa, Homo sapiens
LPPPIGGAGPPLGLPK

SEQ ID NO: 99 - protein, 10 aa, Homo sapiens
LFIGGLSFET

SEQ ID NO: 100 - protein, 6 aa, Artificial Sequence
  synthetic peptide
SISSYY

SEQ ID NO: 101 - protein, 46 aa, Artificial Sequence
  synthetic peptide
GGGGSGGGGSYGYTHLSTGDLLRGLFEAIEGFIENGWEGMIDGWYG

SEQ ID NO: 102 - protein, 39 aa, Artificial Sequence
  synthetic peptide
GGGGSGGGGSLFIGGLSFETPFVIGAGVLGALGTGIGGI

SEQ ID NO: 103 - protein, 48 aa, Artificial Sequence
  synthetic peptide
GGGGSGGGGSGAAAGLFEAIEGFIENGWEGMIDGWYGYTHLSTGDLLR

SEQ ID NO: 104 - protein, 36 aa, Artificial Sequence
  synthetic peptide
GGGGSGGGGSGAAAPFVIGAGVLGALGTGIGGLSFE

SEQ ID NO: 105 - protein, 36 aa, Artificial Sequence
  synthetic peptide
GGGGSGGGGSAAAGLFEAIEGFIENGWEGMIDGWYG

SEQ ID NO: 106 - protein, 33 aa, Artificial Sequence
  synthetic peptide
GGGGSGGGGSGAAAPFVIGAGVLGALGTGIGGI

SEQ ID NO: 107 - protein, 31 aa, Artificial Sequence
  synthetic peptide
GGGSGGGSGLFEAIEGFIENGWEGMIDGWYG

SEQ ID NO: 108 - protein, 27 aa, Artificial Sequence
  synthetic peptide
GGGSGGGSPFVIGAGVLGALGTGIGGI

SEQ ID NO: 109 - protein, 31 aa, Artificial Sequence; identical to SEQ ID NO: 107
  synthetic peptide

SEQ ID NO: 110 - protein, 27 aa, Artificial Sequence
  synthetic peptide
GGGSGGGSPFVIGAGVLGALGTGIGGI

SEQ ID NO: 111 - protein, 31 aa, Artificial Sequence; identical to SEQ ID NO: 107
  synthetic peptide

SEQ ID NO: 112 - protein, 27 aa, Artificial Sequence
  synthetic peptide
GGGSGGGSPFVIGAGVLGALGTGIGGI

SEQ ID NO: 113 - protein, 214 aa, Mus musculus
DIQMTQSPSSLSASVGDRVTITCRASQSVAAAVAWYQQKPGKAPKLLIYSASSLYSGVPSRFSGSRSGTDFTLTISSLQPEDFATYYCQQSYWYPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 231 aa, Mus musculus
EVQLVESGGGLVQPGGSLRLSCAASGFNLSSSSIHWVRQAPGKGLEWVASISSYYGYTYYADSVKGRFTISADTSKNTAYLQMNSLRAEDTAVYYCARNDDWYIWDWYYTRWYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGRQTYICNVNHKPSNTKVDKRVEPKSC

SEQ ID NO: 115 - protein, 8 aa, Artificial Sequence
  synthetic peptide
SIINFEKL

SEQ ID NO: 116 - protein, 21 aa, Artificial Sequence
  synthetic peptide
ISQAVHAAHAEINEAGREVVG

SEQ ID NO: 117 - protein, 21 aa, Artificial Sequence
  synthetic peptide
YEEWAYYSSEMAFSIINFEKL

SEQ ID NO: 118 - protein, 34 aa, Artificial Sequence
  synthetic peptide
YEEWAYYSSEMAFISQAVHAAHAEINEAGREVVG

SEQ ID NO: 119 - protein, 5 aa, Artificial Sequence
  synthetic peptide
GGGSC

(19) United States
(12) Patent Application Publication
(10) Pub. No.: US 2021/0171642 A1
(43) Pub. Date: Jun. 10, 2021

(54) METHODS AND COMPOSITIONS FOR TARGETING LIVER AND LYMPH NODE SINUSOIDAL ENDOTHELIAL CELL C-TYPE LECTIN (LSECTIN)

(71) Applicant: The University of Chicago, Chicago, IL (US)

(72) Inventors: Jeffrey HUBBELL, Chicago, IL (US); Elyse A. WATKINS, Chicago, IL (US); Tomasz SLEZAK, Chicago, IL (US)

(73) Assignee: The University of Chicago, Chicago, IL (US)

(21) Appl. No.: 15/733,657

(22) PCT Filed: Mar. 26, 2019
(86) PCT No.: PCT/US2019/024052

§ 371 Date: Sep. 25, 2020

Related U.S. Application Data
(60) Provisional application No. 62/647,911, filed on Mar. 26, 2018.

(51) Int. Cl. C07K 16/28

(52) U.S. Cl. CPC C07K 16/2851; A61K 2039/505; C07K 2317/565

(57) ABSTRACT

Certain embodiments are directed to compositions and methods for targeting an antigen to a liver and lymph node C type lectin (LSECtin). In particular aspects the compositions disclosed herein can induce tolerogenic immunity to the targeted antigen.

### CROSS-REFERENCE TO RELATED APPLICATIONS

This application claims the benefit of priority of U.S. Provisional Patent Application No. 62/647,911 filed Mar. 26, 2018, which is hereby incorporated by reference in its entirety.

### REFERENCE TO SEQUENCE LISTING

The instant application contains a Sequence Listing which has been submitted in ASCII format and is hereby incorporated by reference in its entirety. Said ASCII copy, created on Feb. 11, 2021, is named ARCD.P0653US_Sequence_Listing.txt and is 91,447 bytes in size.

### BACKGROUND

### A. Field

Several embodiments disclosed herein relate generally to the field of medicine and immunology. More specifically, several embodiments relate to targeting lymph node and/or liver sinusoidal endothelial cells C-type lectin (LSECtin) to modulate immunological tolerance via liver sinusoidal endothelial cells (LSEC).

### B. Description of the Related Art

The main functions of the liver are detoxification, metabolism, and production of important substances such as albumin and bile. Liver sinusoidal endothelial cells (LSECs) are highly specialized endothelial cells representing the interface between blood cells on the one side and hepatocytes and hepatic stellate cells on the other side. Despite its commonly appreciated main functions, an underappreciated function of the liver is its role in immunity. The liver is subject to blood-borne pathogens to which it must mount a productive immune response, such as in hepatitis and malaria. The liver is home to the largest population of tissue resident macrophages, Kupffer cells, as well as the largest concentration of Natural Killer cells and Natural Killer T cells (Jenne and Kubes, Nat. Immunol. 14:996-1006, 2013). The anatomy of the liver is suited for immune interactions. Blood slows significantly as it passes through the liver sinusoids, and the endothelium is fenestrated, exposing sub-endothelial cells residing in the space of Disse to circulating cells. Together this allows for intimate interactions between circulating lymphocytes and cells in the sinusoids, including hepatocytes, dendritic cells, Kupffer cells, and liver sinusoidal endothelial cells (LSECs).

Immune responses are necessary for protection against potentially pathogenic microorganisms. However, undesired immune activation can cause injurious processes leading to damage or destruction of one's own tissues. Undesired immune activation occurs, for example, in autoimmune diseases where antibodies and/or T lymphocytes react with self-antigens to the detriment of the body's tissues. This is also the case in allergic reactions characterized by an exaggerated immune response to certain environmental matters and which may result in inflammatory responses leading to tissue destruction, as well as in rejection of transplanted organs mediated by alloreactive T cells present in the host.

Immune tolerance is the acquired lack of specific immune responsiveness to an antigen to which an immune response would normally occur. Typically, to induce tolerance, there must be an exposure to a tolerizing antigen, which results in the death or functional inactivation of certain lymphocytes. This process generally accounts for tolerance to self-antigens, or self-tolerance. Immunosuppressive agents are useful in prevention or reduction of undesired immune responses, e.g., in treating patients with autoimmune diseases or with allogeneic transplants. However, immunosuppressive agents can also cause systemic immune suppression, toxicity and even death due to opportunistic infections.

There is a need for additional compositions and methods for inducing immune tolerance, especially antigen-specific immune tolerance.

### SUMMARY

Embodiments described herein address the unmet need in inducing immune tolerance by targeting the liver (e.g., LSECs) for purposes of modulating the immune system. Despite the liver's important role in eliciting an immune response, the tolerogenic nature of the liver is under investigation. Among the first reports of a tolerogenic role of the liver was in transplants. It was found that transplanted livers were accepted across Major Histocompatibility Complex (MHC) barriers in the absence of immunosuppression (Caine et al., 1969). It was later found that injection of allogeneic cells into the portal vein resulted in tolerance to alloantigens (Qian et al., The Journal of Immunology, 1985; Fujiwara et al., The Journal of Immunology, 1986; Yamamoto et al. Immunobiology, 1997). Other studies have found that antigen injected into the portal vein, but not into systemic circulation, induces tolerance (Cantor and Dumont, Nature 215:744-45, 1967). Thus, it appears that under certain circumstances the immune system mounts a productive immune response, while under other circumstances it tolerizes the immune system.

LSECs are involved in the dichotomy between immunity and tolerance. LSECs have been shown to be extremely efficient at endocytosis (Magnusson and Berg, Biochem. J. 257:651-56, 1989). These cells express components necessary for T cell activation, including MHC class II and co-stimulatory molecules (Lohse et al., Gastroenterology 110:1175-81, 1996). LSECs can process and present antigen to CD4+ T cells, but they may also cross present antigen to CD8 T cells, a capability that has otherwise only been described in dendritic cells and a subset of macrophages (Knolle et al., Gastroenterology 116:1428-40, 1999; Limmer et al., Nat. Med. 6:1348-54, 2000). LSECs express Toll-like receptors (TLRs) that enable them to mount productive immune responses. Upon stimulation by a variety of pathogen associated molecular patterns, they may produce immunogenic cytokines such as interferon beta, interleukin 6, and interleukin 12, and may activate CD8+ T cells as well as control hepatitis B virus replication in hepatocytes (Wu et al. Immunology 129:363-74, 2010; Martin-Armas et al. J. Hepatol. 44:939-46, 2006; Wu et al. Hepatology 46:1769-78, 2007; Liu et al. J. Immunol. 191:6178-90, 2013). LSECs also express a variety of endocytic receptors, such as mannose receptor, FCyRIIb, and LSECtin (Magnusson and Berg, Biochem. J. 257:651-56, 1989; Mousavi et al. Hepatology 46:871-84, 2007; Liu et al. J. Biol. Chem. 279:18748-58, 2004). The former two have been shown to mediate traffic to endosomal compartments and lead to antigen presentation, so it is reasonable to assume that LSECtin, given its role in mediating endocytosis, will have a similar function in antigen presentation (Mousavi et al. Hepatology 46:871-84, 2007; Liu et al. J. Biol. Chem. 279:18748-58, 2004). Conversely, under circumstances in which there is no excessive danger signal, LSECs have been shown to present and cross present antigen to efficiently induce CD4+ regulatory T cells and CD8+ T cell deletion (Limmer et al. Nat. Med. 6:1348-54, 2000; Kruse et al. Hepatology 50:1904-13, 2009). It has further been shown that LSECs can in fact deter dendritic cells from inducing immunity in vivo (Schildberg et al. Eur. J. Immunol. 38:957-67, 2008).

As disclosed herein, solutions to the above described problems are provided, for example, by the various compositions and methods described herein for targeting LSECtin. It is believed that LSECs are the only cells that express LSECtin in the liver (Liu et al. J. Biol. Chem. 279:18748-58, 2004). LSECtin is a scavenger receptor that is capable of binding mannose and N-acetyl-glucosamine, and triggers rapid internalization of antigens. Because antigens are quickly delivered to the liver when administered systemically, in several embodiments, antigens targeted to LSECtin can be engulfed exclusively by the many LSECs lining the liver sinusoids, and that this antigen would be presented efficiently to circulating T cells. Prior to the embodiments described herein, it was unknown if this route of antigen presentation would lead to productive immunity or tolerogenic immunity. Described herein is the targeting of antigens to LSECtin to induce tolerogenic immunity, leading to new methods, compositions, and uses thereof for inducing antigen-specific tolerance.

In several embodiments, there is provided herein a composition for induction of antigen-specific tolerance, the composition comprising: a binding moiety that binds to Liver Sinusoidal Endothelial Cell C-Type Lectin (LSECtin) comprising an LSECtin-binding moiety and an antigen to which tolerance is desired. In several embodiments, the LSECtin-binding moiety is specific for human LSECtin. In some embodiments, the LSECtin-binding moiety is cross-reactive with one or more additional species. For example, in one embodiment the LSECtin-binding moiety binds to both primate (cynomolgus) and human LSECtin. In several embodiments, the LSECtin-binding moiety comprises a heavy chain complementarity determining region (CDRH) comprising an amino acid sequence of SISSYY (SEQ ID NO:100). In several embodiments the antigen to which tolerance is desired comprises a full length antigen, while in several embodiments, the antigen comprises one or more antigens or one or more fragments of the one or more antigens. In several embodiments, the antigen to which tolerance is desired is covalently coupled to the LSECtin-binding moiety or joined to the LSECtin-binding moiety via a linker. In several embodiments, when a subject is exposed to the antigen alone, the subject reacts to the antigen alone with an unwanted immune response. However, as disclosed herein, when the subject is exposed to the compositions disclosed herein, the subject has a reduced immune response to a subsequent exposure to the antigen.

Also provided herein are methods for inducing tolerance to a specific antigen in a subject, the method comprising administering to the subject a composition comprising a binding moiety that binds to LSECtin and an antigen to which tolerance is desired. As discussed herein, in several embodiments, the LSECtin binding-moiety comprises a heavy chain complementarity determining region (CDRH) comprising an amino acid sequence of SISSYY (SEQ ID NO:100). In several embodiments, the LSECtin-binding moiety is specific for human LSECtin. In some embodiments, the LSECtin-binding moiety is cross-reactive with one or more additional species. For example, in one embodiment the LSECtin-binding moiety binds to both primate (cynomolgus) and human LSECtin. In several embodiments the antigen to which tolerance is desired comprises a full-length antigen, while in several embodiments, the antigen comprises one or more antigens or one or more fragments of the one or more antigens. In several embodiments, the antigen to which tolerance is desired is covalently coupled to the LSECtin-binding moiety or joined to the LSECtin-binding moiety via a linker. In several embodiments, when a subject is exposed to the antigen alone, the subject reacts to the antigen alone with an unwanted immune response. However, according to the methods and uses disclosed herein, administration of the compositions disclosed herein to the subject results in antigen-specific tolerance being developed to the antigen and as a result, the subject has a reduced immune response to a subsequent exposure to the antigen.

In several embodiments, the LSECtin-binding moiety is an LSECtin-specific antibody or a fragment of an LSECtin-specific antibody. In several embodiments, the LSECtin-binding moiety is fragment of an LSECtin-specific antibody, for example an scFv or a Fab. In addition to the CDRH, in several embodiments, the LSECtin-binding moiety further comprises an additional CDRH comprising an amino acid sequence of SSI. In several embodiments, the CDRH comprises an amino acid sequence of SISSYYX₃YTX₄ (SEQ ID NO:68) and the additional CDRH comprises an amino acid sequence of X₁SX₂SSI (SEQ ID NO:67). In several embodiments, CDRHs having at least about 80% sequence identity to those in SEQ ID NO:67 or 68 are used. In several embodiments, the LSECtin-binding moiety further comprises at least a third CDRH and a light chain complementarity determining region (CDRL). In several embodiments, the CDRH comprises an amino acid sequence of SISSYYGYTY (SEQ ID NO:59) and the additional CDRH comprises an amino acid sequence of LSSSSI (SEQ ID NO:55). In several embodiments, the LSECtin-binding moiety further comprises a light chain complementarity determining region (CDRL) having an amino acid sequence of SYWYPV (SEQ ID NO:51) or a CDRL having at least about 80% sequence identity to SEQ ID NO:51. In several embodiments, the LSECtin-binding moiety further comprises at least a third CDRH having an amino acid sequence of NDDWYIWDWYYTRWYGL (SEQ ID NO:63), or a third CDRH having at least about 80% sequence identity to SEQ ID NO:63. In several embodiments, the LSECtin-binding moiety further comprises one or more additional CDRL.

In some embodiments, the CDRH comprises an amino acid sequence of SISSYYSYTS (SEQ ID NO:12) and the additional CDRH comprises an amino acid sequence of VSYSSI (SEQ ID NO:9) or polypeptides having at least about 80% sequence identity to SEQ ID NO:12 or 9. In several embodiments, the LSECtin-binding moiety further comprises a light chain complementarity determining region (CDRL) having an amino acid sequence of YLAYQSPL (SEQ ID NO:4), or a CDRL having at least about 80% sequence identity to SEQ ID NO:4. In several embodiments, the LSECtin-binding moiety further comprises at least a third CDRH having an amino acid sequence of YEEWAYYSSEMAF (SEQ ID NO:18) or a third CDRH having at least about 80% sequence identity to SEQ ID NO:18. In several embodiments, the LSECtin-binding moiety further comprises one or more additional CDRL.

In several embodiments, the LSECtin-binding moiety has been affinity matured or is subjected to an affinity maturation campaign. In several embodiments, the CDRH and/or CDRL sequences are humanized.

Depending on the embodiment, the antigen to which tolerance is desired can vary. For example, in several embodiments, the antigen (or fragment, or combination of fragments) is associated with one or more of multiple sclerosis, Celiac disease and/or Type I Diabetes.

In several embodiments, the antigen comprises a polypeptide comprising a portion of SEQ ID NO:26. In several embodiments, the antigen comprises a polypeptide comprising a portion of SEQ ID NO:27. In several embodiments, the antigen comprises a polypeptide comprising a portion of SEQ ID NO:28. In several embodiments, the antigen comprises a polypeptide comprising a portion of SEQ ID NO:26 and a portion of SEQ ID NO:27 and/or a portion of SEQ ID NO:28. In several embodiments, the antigen comprises a polypeptide comprising SEQ ID NO:69 or a polypeptide having at least about 85% sequence identity thereto and SEQ ID NO:70 or a polypeptide having at least about 85% sequence identity thereto. In several embodiments, the antigen comprises a polypeptide comprising SEQ ID NO:71 or a polypeptide having at least about 85% sequence identity thereto and SEQ ID NO:75 or a polypeptide having at least about 85% sequence identity thereto. In several embodiments, the antigen comprises a polypeptide comprising SEQ ID NO:72 or a polypeptide having at least about 85% sequence identity thereto and SEQ ID NO:76 or a polypeptide having at least about 85% sequence identity thereto. In several embodiments, the antigen comprises a polypeptide comprising SEQ ID NO:73 or a polypeptide having at least about 85% sequence identity thereto and SEQ ID NO:35 or a polypeptide having at least about 85% sequence identity thereto. In several embodiments, the antigen comprises a polypeptide comprising a portion of SEQ ID NO:26, a portion of SEQ ID NO:27, and a portion of SEQ ID NO:28. In several embodiments, the antigen comprises a polypeptide comprising one or more of SEQ ID NO:35, SEQ ID NO:69, SEQ ID NO:70, SEQ ID NO:71, and SEQ ID NO:72, or a polypeptide having at least about 85% sequence identity to any of SEQ ID NO:35, SEQ ID NO:69, SEQ ID NO:70, SEQ ID NO:71, or SEQ ID NO:72. In several embodiments, the antigen comprises a polypeptide comprising one or more of SEQ ID NO:73, SEQ ID NO:74, SEQ ID NO:75, SEQ ID NO:76, and SEQ ID NO:35, or a polypeptide having at least about 85% sequence identity to any of SEQ ID NO:73, SEQ ID NO:74, SEQ ID NO:75, SEQ ID NO:76, or SEQ ID NO:35. In several embodiments, the antigen comprises a polypeptide comprising one or more of SEQ ID NO:35, SEQ ID NO:75, SEQ ID NO:76, SEQ ID NO:71, SEQ ID NO:73, SEQ ID NO:74, and SEQ ID NO:72, or a polypeptide having at least about 85% sequence identity to any of SEQ ID NO:35, SEQ ID NO:75, SEQ ID NO:76, SEQ ID NO:71, SEQ ID NO:73, SEQ ID NO:74, or SEQ ID NO:72. In several embodiments, the antigen comprises a polypeptide comprising one or more of the amino acids sequences of SEQ ID NO:29, SEQ ID NO:30, SEQ ID NO:31, SEQ ID NO:32, SEQ ID NO:33, SEQ ID NO:34 and SEQ ID NO:35. In several embodiments, the antigen comprises a polypeptide comprising an amino acid sequence of SEQ ID NO:29. In several embodiments, the antigen comprises a polypeptide comprising an amino acid sequence of SEQ ID NO:30. In several embodiments, the antigen comprises a polypeptide comprising an amino acid sequence of SEQ ID NO:31. In several embodiments, the antigen comprises a polypeptide comprising an amino acid sequence of SEQ ID NO:32. In several embodiments, the antigen comprises a polypeptide comprising an amino acid sequence of SEQ ID NO:33. In several embodiments, the antigen comprises a polypeptide comprising an amino acid sequence of SEQ ID NO:34. In several embodiments, the antigen comprises a polypeptide comprising an amino acid sequence of SEQ ID NO:35. In several embodiments, the antigen comprises a polypeptide comprising an amino acid sequence of SEQ ID NO:42 or SEQ ID NO:43, or a polypeptide having at least about 85% sequence identity to any of SEQ ID NO:42 or SEQ ID NO:43. In several embodiments, the antigen comprises a polypeptide comprising an amino acid sequence of SEQ ID NO:77, SEQ ID NO:78 or SEQ ID NO:79, or a polypeptide having at least about 85% sequence identity to any of SEQ ID NO:77, SEQ ID NO:78, or SEQ ID NO:79. In several embodiments, the antigen comprises a polypeptide comprising a portion of SEQ ID NO:23. In several embodiments, the antigen comprises a polypeptide comprising an amino acid sequence comprising a portion of SEQ ID NO:23 and a portion of SEQ ID NO:80. In several embodiments, the antigen comprises one or more polypeptides selected from the group consisting of SEQ ID NO:91, SEQ ID NO:92, SEQ ID NO:93, SEQ ID NO:94, SEQ ID NO:95, and SEQ ID NO:96, or a polypeptide having at least about 85% sequence identity to any of SEQ ID NO:91, SEQ ID NO:92, SEQ ID NO:93, SEQ ID NO:94, SEQ ID NO:95 or SEQ ID NO:96. In several embodiments, the antigen comprises one or more polypeptides selected from the group consisting of SEQ ID NO:82, SEQ ID NO:83, SEQ ID NO:84, SEQ ID NO:85, SEQ ID NO:86, SEQ ID NO:87, SEQ ID NO:88, SEQ ID NO:89, and SEQ ID NO:90, or a polypeptide having at least about 85% sequence identity to any of SEQ ID NO:82, SEQ ID NO:83, SEQ ID NO:84, SEQ ID NO:85, SEQ ID NO:86, SEQ ID NO:87, SEQ ID NO:88, SEQ ID NO:89 or SEQ ID NO:90.

In several embodiments, the antigen comprises one or more of high molecular weight glutenin; low molecular weight glutenin; alpha-, gamma- and omega-gliadin; hordein; secalin; avenin; a portion of any of the antigens, and a mimetic of any of antigens.

In several embodiments, the antigen comprises one or more of gliadin, a portion of gliadin, and a mimetic of any of the antigens.

In several embodiments, the antigen comprises one or more of insulin, proinsulin, preproinsulin, glutamic acid decarboxylase-65 (GAD-65), GAD-67, insulinoma-associated protein 2 (IA-2), and insulinoma-associated protein 213 (IA-213), ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, SST G-protein coupled receptors 1-5, and a portion of any of the antigens, and a mimetic of any of the antigens.

In several embodiments, the antigen comprises one or more of myelin basic protein, myelin oligodendrocyte glycoprotein and proteolipid protein, a portion of any of the antigens, and a mimetic of any of the antigens.

In several embodiments, the antigen comprises one or more of Abciximab, Adalimumab, Agalsidase alfa, Agalsidase beta, Aldeslukin, Alglucosidase alfa, Factor VIII, Factor IX, Infliximab, L-asparaginase, Laronidase, Natalizumab, Octreotide, Phenylalanine ammonia-lyase (PAL), or Rasburicase (uricase), a portion of any of the antigens, and a mimetic of any of the antigens.

In several embodiments, the antigen comprises one or more subunits of the MHC class I and MHC class II haplotype proteins, and minor blood group antigens RhCE, Kell, Kidd, Duffy and Ss.

In several embodiments, the antigen comprises one or more of insulin, proinsulin, preproinsulin, a tolerogenic portion of any of the antigens, or a mimetic of any one of the antigens.

Also provided herein are methods of inducing tolerance to a specific antigen in a subject comprising administering to the subject tolerogenic compounds disclosed herein. Antigens associated with multiple sclerosis can be used in a composition and method for treating multiple sclerosis in a subject. Antigens associated with Celiac disease can be used in a composition and method for treating Celiac disease in a subject. Antigens associated with food allergy can be used in a composition and method for treating food allergy in a subject. Antigens associated with Type 1 diabetes can be used in a composition and method for treating Type 1 diabetes in a subject.

There are also provided herein uses of the compounds disclosed herein for inducing tolerance to a specific antigen in a subject. Also provided herein are uses of the compounds disclosed herein for the preparation of a medicament for inducing tolerance to a specific antigen in a subject.

Certain embodiments are directed to LSECtin binding moieties that specifically bind LSECtin. In certain aspects the LSECtin binding moieties are antibodies, LSECtin binding fragments (e.g., Fabs), or portions of antibodies (e.g., CDRs) that specifically bind to LSECtin. In several embodiments, the LSECtin binding moieties are operatively coupled to an antigen for the purpose of delivering the antigen to LSECs. Depending on the embodiment, an antigen (or antigens), a fragment of an antigen (e.g., an immunogenic portion of an antigen), and/or a mimotope of an antigen, either in purified forms or cell-derived forms such as exosomes, cell fragments, or cells, may be operatively linked to the LSECtin binding moiety (e.g., LSECtin binding antibody or LSECtin binding fragment thereof) to form a LSEC targeting complex. The LSEC targeting complex can be used to induce immunological tolerance to an antigen that is included in the LSEC targeting complex and is delivered to LSECs.

In certain embodiments an LSECtin binding moiety (LBM), such as an antibody or antibody fragment, is conjugated to antigen X forming a LBM complex having the formula [A-B-X], where A is an LSECtin binding moiety; B is an optional linker; and X is a foreign transplant antigen, or alloantigen, or autoimmune antigen, or a fragment(s) of any such antigens. In several embodiments, the antigen can be an antigen (or fragment(s)) against which a subject, such as a transplant recipient or autoimmune patient, develops an unwanted immune response. In several embodiments, the antigen can be a foreign extracellular vesicle, cell fragment, or cell containing alloantigens against which transplant recipients or autoimmune patients develop and unwanted immune response. In still further embodiments, the antigen can be a foreign food, animal, plant or environmental antigen (or fragment(s) thereof) against which patients develop an unwanted immune response. In certain aspects the antigen can be a foreign therapeutic agent (or fragment(s) thereof) against which patients develop an unwanted immune response. In a further aspect the antigen can be a synthetic self-antigen (or fragments(s) thereof) to which patients develop an unwanted immune response. In several embodiments, the antigen can be a tolerogenic (e.g., immunogenic) portion of a larger antigen. In certain embodiments, an antigen or antigen portion is, is at least, or is at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403,404, 405,406, 407,408, 409,410, 411, 412,413, 414,415, 416, 417,418, 419,420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids in length (or any range derivable therein). In several embodiments an LSECtin binding moiety (LBM), such as an antibody or antibody fragment, is conjugated to a plurality of antigens, or antigenic fragments (e.g., X1, X2, X3, Xn) forming a LBM complex having the formula [A-B-X₁-B-X2-B-X3-B-Xn], where A is an LSECtin binding moiety; B is an optional linker; and X₁, X2, X3, and Xn are antigens as disclosed herein. Depending on the embodiment, X₁, X2, X3 etc. may be the same, or different, antigens. Additionally, X1, X2, X3, etc., may be a fragment derived from a different portion of an antigen of interest, for example a first, second, and third immunogenic region (overlapping in some embodiments) of a larger antigen of interest.

As used herein an “antigen-binding molecule (ABM)” relates to molecules, in particular proteins such as antibodies, which contain antibody variable regions that provide specific binding to an epitope or portion of an antigen. The antibody variable region can be present in, for example, a complete antibody, an antibody fragment, and a recombinant derivative or analog of an antibody or antibody fragment. The term “antigen-binding fragment” of an antibody (or “binding portion”), as used herein, refers to one or more fragments of an antibody that retain the ability to specifically bind an antigen. Antigen-binding fragments containing antibody variable regions include, but are not limited to “Fv,” “Fab,” and “F(ab′)₂” regions, “single domain antibodies (sdAb),” “nanobodies,” “single chain Fv (scFv)” fragments, “tandem scFvs” (V_{H}A-V_{L}A-V_{H}B-V_{L}B), “diabodies,” “triabodies” or “tribodies,” “single-chain diabodies (scDb),” and “bi-specific T-cell engagers (BiTEs)”. Antigen-binding molecules can also be antibodies of nonhuman origin, such as camelid antibodies. In certain embodiments, the antigen binding molecule is not a complete antibody but is less than full length. In certain embodiments, the antigen binding molecule is a humanized antigen binding molecule.

In certain embodiments, an LSECtin binding moiety is, is at least, or is at most 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids in length (or any range derivable therein).

In some embodiments, a linker is, is at least, or is at most 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids (or any range derivable therein). The linker can be a synthetic linker in certain embodiments.

In other embodiments, there is a nucleic acid encoding all or part of a polypeptide, e.g., a LSECtin targeting complex. In further embodiments, the nucleic acid is in a plasmid or vector or expression construct. In additional embodiments, the nucleic acid is in a recombinant host cell.

Other embodiments of the invention are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

The term “about” when used in connection with a numerical value is meant to encompass numerical values within a range typically having a lower limit that is, e.g., 5-10% smaller than the indicated numerical value and having an upper limit that is, e.g., 5-10% larger than the indicated numerical value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics.

A “chemical modification” refers to a change in the naturally-occurring chemical structure of one or more amino acids of a polypeptide. Such modifications can be made to a side chain or a terminus, e.g., changing the amino-terminus or carboxyl terminus. In some embodiments, the modifications are useful for creating chemical groups that can conveniently be used to link the polypeptides to other materials, or to attach a therapeutic agent.

“Conservative changes” can generally be made to an amino acid sequence without altering activity. These changes are termed “conservative substitutions” or mutations; that is, an amino acid belonging to a grouping of amino acids having a particular size or characteristic can be substituted for another amino acID Substitutes for an amino acid sequence can be selected from other members of the class to which the amino acid belongs. For example, the nonpolar (hydrophobic) amino acids include alanine, leucine, isoleucine, valine, proline, phenylalanine, tryptophan, methionine, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such substitutions are not expected to substantially affect apparent molecular weight as determined by polyacrylamide gel electrophoresis or isoelectric point. Conservative substitutions also include substituting optical isomers of the sequences for other optical isomers, specifically d amino acids for 1 amino acids for one or more residues of a sequence. Moreover, all of the amino acids in a sequence can undergo a d to 1 isomer substitution. Exemplary conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free —NH₂. Yet another type of conservative substitution constitutes the case where amino acids with desired chemical reactivities are introduced to impart reactive sites for chemical conjugation reactions, if the need for chemical derivatization arises. Such amino acids include but are not limited to Cys (to insert a sulfhydryl group), Lys (to insert a primary amine), Asp and Glu (to insert a carboxylic acid group), or specialized noncanonical amino acids containing ketone, azide, alkyne, alkene, and tetrazine side-chains. Conservative substitutions or additions of free —NH₂ or —SH bearing amino acids can be particularly advantageous for chemical conjugation of Fabs to antigens or vesicles. Moreover, point mutations, deletions, and insertions of the polypeptide sequences or corresponding nucleic acid sequences can in some cases be made without a loss of function of the polypeptide or nucleic acid fragment. Substitutions can include, e.g., 1, 2, 3, 4, 5, 6, 7, 8, 9, 10, 15, 20, 25, 30, 35, 40, 45, 50 or more residues (including any number of substitutions between those listed). A variant usable in certain embodiments may exhibit a total number of up to 100 (e.g., up to 1, 2, 3, 4, 5, 6, 7, 8, 9, 10, 15, 20, 25, 30, 35, 40, 45, 50, 55, 60, 65, 70, 75, 80, 85, 90, 95, or 100, including any number in between those listed) changes (e.g., exchanges, insertions, deletions, N-terminal truncations, and/or C-terminal truncations) in the in the amino acid or nucleotide sequence per, for example every 500 amino acids or nucleotides. In several embodiments, the number of changes is greater than 100 while maintaining the desired character (e.g., function or antigenic nature) of the polypeptide. Additionally, in several embodiments, the variants include polypeptide sequences or corresponding nucleic acid sequences that exhibit a degree of functional equivalence with a reference (e.g., unmodified or native sequence). In several embodiments, the variants exhibit about 80%, about 85%, about 90%, about 95%, about 97%, about 98%, about 99% functional equivalence to an unmodified or native reference sequence (and any degree of functional equivalence between those listed, including endpoints). The amino acid residues described herein employ either the single letter amino acid designator or the three-letter abbreviation in keeping with the standard polypeptide nomenclature. All amino acid residue sequences are represented herein by formulae with left and right orientation in the conventional direction of amino-terminus to carboxy-terminus.

The terms “effective amount” or “therapeutically effective amount” refer to that amount of a composition of the disclosure that is sufficient to effect treatment, as defined herein, when administered to a mammal in need of such treatment. This amount will vary depending upon the subject and disease condition being treated, the weight and age of the subject, the severity of the disease condition, the particular composition of the disclosure chosen, the dosing regimen to be followed, timing of administration, manner of administration and the like, all of which can readily be determined by one of ordinary skill in the art.

The “numerical values” and “ranges” provided for the various substituents are intended to encompass all integers within the recited range. For example, when defining n as an integer representing a mixture including from about 1 to 100, where the mixture typically encompasses the integer specified as n f about 10% (or for smaller integers from 1 to about 25, 13), it should be understood that n can be an integer from about 1 to 100 (e.g., 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110, or any between those listed) The combined terms “about” and “±10%” or “±3” should be understood to disclose and provide specific support for equivalent ranges wherever used.

The term “optional” or “optionally” means that the subsequently described event or circumstance may or may not occur, and that the description includes instances where said event or circumstance occurs and instances in which it does not.

A peptide, protein, or fragment that specifically binds a particular target is referred to as a “ligand” for that target.

A “polypeptide” is a term that refers to a chain of amino acid residues, regardless of post-translational modification (e.g., phosphorylation or glycosylation) and/or complexation with additional polypeptides, and/or synthesis into multi-subunit complexes with nucleic acids and/or carbohydrates, or other molecules. Proteoglycans therefore also are referred to herein as polypeptides. A long polypeptide (having over about 50 amino acids) is referred to as a “protein.” A short polypeptide (having fewer than about 50 amino acids) is referred to as a “peptide.” Depending upon size, amino acid composition and three dimensional structure, certain polypeptides can be referred to as an “antigen-binding molecule,” “antibody,” an “antibody fragment” or a “ligand.” Polypeptides can be produced by a number of methods, many of which are well known in the art. For example, polypeptides can be obtained by extraction (e.g., from isolated cells), by expression of a recombinant nucleic acid encoding the polypeptide, or by chemical synthesis. Polypeptides can be produced by, for example, recombinant technology, and expression vectors encoding the polypeptide introduced into host cells (e.g., by transformation or transfection) for expression of the encoded polypeptide.

As used herein, “pharmaceutically acceptable carrier” or “pharmaceutically acceptable excipient” includes any and all solvents, dispersion media, coatings, antibacterial and antifungal agents, isotonic and absorption delaying agents and the like. In several embodiments, these media and agents can be used in combination with pharmaceutically active substances. Except insofar as any conventional media or agent is incompatible with the active ingredient, its use in the therapeutic compositions is contemplated. Supplementary active ingredients can also be incorporated into the compositions.

The term “purified” as used herein with reference to a polypeptide refers to a polypeptide that has been chemically or biologically synthesized and is thus substantially uncontaminated by other polypeptides, or has been separated or isolated from most other cellular components by which it is naturally accompanied (e.g., other cellular proteins, nucleic acids, or cellular components such as lipid membrane). An example of a purified polypeptide is one that is at least 70%, by dry weight, free from the proteins and naturally occurring organic molecules with which it naturally associates. A preparation of a purified polypeptide therefore can be, for example, at least 80%, at least 90%, or at least 99%, by dry weight, the polypeptide. Polypeptides also can be engineered to contain a tag sequence (e.g., a polyhistidine tag, a myc tag, a FLAG® tag, a SNAP® tag, or other affinity tag) that facilitates purification or marking (e.g., capture onto an affinity matrix, visualization under a microscope). Thus a purified composition that comprises a polypeptide refers to a purified polypeptide unless otherwise indicated. The term “isolated” indicates that the polypeptides or nucleic acids of the disclosure are not in their natural environment. Isolated products of the disclosure can thus be contained in a culture supernatant, partially enriched, produced from heterologous sources, cloned in a vector or formulated with a vehicle, etc.

The term “sequence identity” is used with regard to polypeptide sequence comparisons. This expression in particular refers to a percentage of sequence identity, for example at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Particularly, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide.

“Specific binding,” as that term is commonly used in the biological arts, refers to a molecule that binds to a target with a relatively high affinity as compared to non-targets, and generally involves a plurality of non-covalent interactions, such as electrostatic interactions, van der Waals interactions, hydrogen bonding, and the like. Specific binding interactions characterize antibody-antigen binding, enzyme-substrate binding, and certain protein-receptor interactions; while such molecules might bind tissues besides their specific targets from time to time, to the extent that such non-target binding is inconsequential, the high-affinity binding pair can still fall within the definition of specific binding.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The term “unwanted immune response” refers to a reaction by the immune system of a subject, which in the given situation is not desirable. The reaction of the immune system is unwanted if such reaction does not lead to the prevention, reduction, or healing of a disease or disorder but instead causes, enhances or worsens a disorder or disease. Typically, a reaction of the immune system causes, enhances or worsens a disease if it is directed against an inappropriate target. By way of non-limiting example, an unwanted immune response includes but is not limited to transplant rejection, immune response against a therapeutic agent, autoimmune disease, and allergy or hypersensitivity.

The term “variant” is to be understood as a protein that differs in comparison to the protein from which it is derived by one or more changes in its length, sequence, or structure. The polypeptide from which a protein variant is derived is also known as the parent polypeptide or polynucleotide that genetically encodes the polypeptide. The term “variant” comprises “fragments” or “derivatives” of the parent molecule. Typically, “fragments” are smaller in length or size than the parent molecule, whilst “derivatives” exhibit one or more differences in their sequence or structure in comparison to the parent molecule. Also encompassed modified molecules such as but not limited to post-translationally modified proteins (e.g., glycosylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Naturally occurring and artificially constructed variants are to be understood to be encompassed by the term “variant” as used herein. Further, the variants usable in severable embodiments may also be derived from homologs, orthologs, or paralogs of the parent molecule or from artificially constructed variant, provided that the variant exhibits at least one biological activity of the parent molecule, i.e., is functionally active. A variant can be characterized by a certain degree of sequence identity to the parent polypeptide from which it is derived. More precisely, a protein variant in the context of the present disclosure may exhibit at least 80% sequence identity to its parent polypeptide. In several embodiments, the sequence identity of protein variants is over a continuous stretch of 20, 30, 40, 45, 50, 60, 70, 80, 90, 100 or more amino acids. As discussed above, in several embodiments variants exhibit about 80%, about 85%, about 90%, about 95%, about 97%, about 98%, about 99% functional equivalence to an unmodified or native reference sequence (and any degree of functional equivalence between those listed).

The term “operatively linked” refers to a situation where two components are combined to form the active complex prior to binding at the target site. For example, a molecule conjugated to one-half of a biotin-streptavidin complex and an antigen complexed to the other one-half of the biotin-streptavidin complex are operatively linked through complexation of the biotin and streptavidin molecules. The term operatively linked is also intended to refer to covalent or chemical linkages that conjugate two molecules together.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the embodiments disclosed herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description.

### DESCRIPTION OF THE DRAWINGS

The following drawings form part of the present specification and are included to further demonstrate certain non-limiting aspects of the disclosed embodiments. Such embodiments may be better understood by reference to one or more of these drawings in combination with the detailed description of the specification embodiments presented herein.

FIG. 1. KingFisher plate layout used for phage display. The first row included streptavidin beads (Dynabeads) with 300 nM, 150 nM, 75 nM, or 20 nM biotinylated LSECtin variants, corresponding to the second, third, fourth, and fifth rounds of display. The next well row contained phage and 2 μM biotinylated SNAP protein, to remove potential SNAP binders. The following row contained 1 μM biotin to saturate biotin sites on the streptavidin beads. The following 4 rows contained 1 μM SNAP to wash. The last row contained thrombin, to elute phage. All steps contained TBS +10 mM CaCl₂.

FIG. 2. Fab hits from phage display. After sequencing approximately 96 single phage from display against each of three variants of LSECtin, numerous converging sequences were identified. The CDRL3, CDRH1, CDRH2, and CDRH3 of selected sequences is provided in FIG. 2, as well as how many times the same sequence appeared in the screening. CDRL3 having an amino acid sequence of SEQ ID NO:3, SEQ ID NO:4, SEQ ID NO:5, SEQ ID NO:6, or SEQ ID NO:7; CDRH1 having an amino acid sequence of SEQ ID NO:8, SEQ ID NO:9, or SEQ ID NO:10; CDRH2 having an amino acid sequence of , SEQ ID NO:11, SEQ ID NO:12, SEQ ID NO:13, SEQ ID NO:14, SEQ ID NO:15, or SEQ ID NO:16; and CDRH3 having an amino acid sequence of SEQ ID NO:17, SEQ ID NO:18 (also referred to in shorthand notation as “YEE”), SEQ ID NO:19, SEQ ID NO:20, SEQ ID NO:21, or SEQ ID NO:22. CDRL3 having an amino acid sequence of SEQ ID NO:51, SEQ ID NO:52, SEQ ID NO:53, or SEQ ID NO:54; CDRH1 having an amino acid sequence of SEQ ID NO:55, SEQ ID NO:56, SEQ ID NO:57, or SEQ ID NO:58; CDRH2 having an amino acid sequence of SEQ ID NO:59, SEQ ID NO:60, SEQ ID NO:61, or SEQ ID NO:62; and CDRH3 having an amino acid sequence of SEQ ID NO:63 (also referred to in shorthand notation as “A1A1”), SEQ ID NO:64, SEQ ID NO:65, or SEQ ID NO:66.

FIG. 3. Flow cytometry data for Fab binding to LSECtin immobilized on streptavidin polystyrene beads. SNAP-LSECtin was biotinylated and bound to Avidin polystyrene beads. Fab was added and detecting with anti-F(ab′)₂ secondary antibody. Dotted line is an irrelevant antibody, solid line is the YEE (SEQ ID NO:18) containing Fab.

FIGS. 4A-B. Fab sequences. Full sequence of non-limiting embodiments of anti-LSECtin Fabs. (A) depicts selected CDR sequences from the Fab referred to as YEE. (B) depicts selected CDR sequences from the Fab referred to as A1A1.

FIGS. 5A-B. Flow cytometry of Fab binding to primary murine LSECs. (A) Liver cells were isolated as described above and stained for CD31 and Stabilin 2, markers sufficient to specifically identify LSECs. (B) Fab bound to LSECs but not to other populations of cells isolated.

FIG. 6. ELISA. Enzyme-linked immunosorbent assay for Fab binding to LSECtin.

FIGS. 7A-C. Immunofluorescence of anti-LSECtin Fab binding to liver sections. (A) Mouse sections stained with stabilin 2 and A1A1 and imaged with 60× magnification. (B) Mouse sections stained with A1A1 and imaged with 20× magnification. (C) Monkey sections stained with A1A1 and imaged with 60× magnification.

FIGS. 8A-B. Uptake analysis of anti-LSECtin Fab. (A) Fab was recombinantly expressed with mCherry on the heavy chain. Fab-mCherry was added to LSECs at 4C for 20 minutes and washed to remove excess Fab. LSECs were incubated at 37C to allow for endocytosis and subsequently stained with an anti-Fab antibody. (B) LSECs were incubated with A1A1-mCherry or irrelevant Fab-mCherry 2 hours at 37C and the fluorescence intensity of mCherry was measured by flow cytometry.

FIGS. 9A-B. Biodistribution of anti-LSECtin Fab in vivo. 25 ng of Fab-800 were injected in vivo (A), and fluorescence was measured in nude mice for 24 hours (IVIS, Perkin Elmer). (B) To measure uptake specifically by LSECs, Fabs were conjugated to the fluorescent dye DY-649 (Dyomics). 2.5 ng of Fab-649 were injected into mice, and mice were sacrificed 30 minutes after injection. LSECs were isolated as in Example 7 and analyzed by flow cytometry for mean fluorescence intensity of Fab.

FIGS. 10A-B. Cathepsin cleaveable linkers between Fab and payload. Figure demonstrates that (A) Fab-CtsL 1 -mCherry is efficiently cleaved by cathepsin L at pH of 6, but not at a pH of 7.5 (B) Fab-mCherry without a linker is not cleaved at pH 6.

FIGS. 11A-B. Results of in vivo tolerance study to model antigens. Data depicted in the Figure demonstrate tolerance to antigens in vivo. (A) Percentage of CD45.1+ OTI or OTII cells in lymph node. (B) Production of interferon gamma after restimulation with SIINFEKL (SEQ ID NO:115) or ISQ.

### DESCRIPTION

The present disclosure provides certain therapeutic compositions (and method of using such compositions) that target the liver, for example, several embodiments target LSEC C-type lectin (LSECtin), a protein found primarily on LSECs in the liver (Liu et al., J. Biol. Chem. 279:18748-58, 2004). Targeting of these compositions to LSECs, according to several embodiments, is accomplished by a high affinity binding moiety, e.g., antibody (whether human or nonhuman or analog thereof, such as camelid), a high affinity fragment antibody (Fab) or related IgG or related single chain variable fragment (scFv) that binds specifically and with high affinity to LSECtin. In several embodiments, the Fab (and/or a related form) can be chemically conjugated or recombinantly expressed as a fusion with an antigen (or an immunogenic fragment, or fragments, of an antigen). In several embodiments, the antigen can be endogenous (a self-antigen) or exogenous (a foreign antigen), including but not limited to: a foreign transplant antigen against which transplant recipients develop an unwanted immune response (e.g., transplant rejection), an extracellular vesicle, cell fragment, or cell containing antigens against which transplant recipients develop an unwanted immune response (e.g., transplant rejection), a foreign food, animal, plant or environmental antigen to which patients develop an unwanted immune response (e.g., allergy or hypersensitivity), a therapeutic agent to which patients develop an unwanted immune response (e.g., hypersensitivity and/or reduced therapeutic activity), a self-antigen to which patients develop an unwanted immune response (e.g., autoimmune disease), or a portion (e.g., a fragment or an epitope) thereof. In several embodiments, these compositions are useful for inducing tolerance to the antigen. As discussed above, a full-length antigen need not be used, rather, in several embodiments, an immunogenic fragment, or fragments, of an antigen are used. One of ordinary skill in the art would readily be able to, without undue experimentation, determine whether a given fragment, or fragments, of a larger antigen would be immunogenic (e.g., able to induce tolerance when administered with compositions according to embodiments disclosed herein).

In additional embodiments, the LSEC-targeting polypeptide can be conjugated to an antibody, antibody fragment, or ligand that binds (e.g., specifically) a circulating protein or peptide or antibody that is causatively involved in transplant rejection, immune response against a therapeutic agent, autoimmune disease, and/or allergy (as discussed above). In several embodiments, these compositions are useful for clearing and/or inducing tolerance to the circulating protein, peptide, or antibody. Accordingly, in line with several embodiments disclosed herein, the compositions of the present disclosure can be used for treating an unwanted immune response, e.g., transplant rejection, an immune response against a therapeutic agent, an autoimmune disease, and/or an allergy.

### I. LSECtin BINDING MOIETIES

An “LSECtin-binding molecule” as used herein relates to molecules, in particular to proteins such as antibodies, which contain antibody regions (e.g., variable regions) that provide specific binding to an epitope, or portion of LSECtin. The antibody variable region can be present in, for example, a complete antibody, an antibody fragment, and a recombinant derivative of an antibody or antibody fragment, or an analog thereof. The term “LSECtin-binding fragment” of an antibody (or “binding portion”), as used herein, refers to one or more fragments of an antibody that retain the ability to specifically bind LSECtin. LSECtin-binding fragments containing antibody variable regions include (without limitation) “Fv,” “Fab,” and “F(ab')2” regions, “single domain antibodies (sdAb),” “nanobodies,” “single chain Fv (scFv)” fragments, “tandem scFvs” (V_{H}A-V_{L}A-V_{H}B-V_{L}B), “diabodies,” “triabodies” or “tribodies,” “single-chain diabodies (scDb),” and “bi-specific T-cell engagers (BiTEs),” as well other protein scaffolds (i.e., analogs) that can support antibody variable regions and maintain their binding specificity. LSECtin-binding molecules can also be antibodies of nonhuman origin, such as camelid antibodies. These include human, non-human (such as mouse) and non-natural (i.e., engineered) proteins, antibodies, chimeric antibodies, humanized antibodies, camelid antibodies, and non-antibody binding scaffolds, such as protein frameworks including complementary determining regions such as fibronectins, knottins, anticalins, affibodies, 4-helix bundle proteins, ankyrin repeat proteins (e.g., DARPins), tetranectins, adnectins, A-domain proteins, lipocalins, immunity protein ImmE7, cytochrome b562, amyloid β-protein precursor inhibitor, cellulose binding domain from cellobiohydrolase Cel7A, carbohydrate binding module CBM4-2; RNA; DNA aptamers; and molecularly imprinted nanoparticles.

In certain aspects the LSECtin binding moiety is an antibody. In a particular aspect the antibody can have a light chain having a light chain amino acid sequence of SEQ ID NO:1 or SEQ ID NO:113 and/or a heavy chain amino acid sequence of SEQ ID NO:2 or SEQ ID NO:114. One non-limiting example of a LSECtin binding moiety is an antibody having one or more CDRs. In a particular embodiment an antibody or LSECtin binding moiety can comprise 1, 2, 3, 4, 5, or 6 CDRs selected from CDRL3 having an amino acid sequence of SEQ ID NO:51, SEQ ID NO:52, SEQ ID NO:53, or SEQ ID NO:54; CDRH1 having an amino acid sequence of SEQ ID NO:55, SEQ ID NO:56, SEQ ID NO:57, or SEQ ID NO:58; CDRH2 having an amino acid sequence of SEQ ID NO:59, SEQ ID NO:60, SEQ ID NO:61, or SEQ ID NO:62; and/or a CDRH3 having an amino acid sequence of SEQ ID NO:63, SEQ ID NO:64, SEQ ID NO:65, or SEQ ID NO:66. In a particular embodiment an antibody or LSECtin binding moiety can comprise 1, 2, 3, 4, 5, or 6 CDRs selected from CDRL1 having an amino acid sequence of SEQ ID NO:49; CDRL2 having an amino acid sequence of SEQ ID NO:50; CDRL3 having an amino acid sequence of SEQ ID NO:3, SEQ ID NO:4, SEQ ID NO:5, SEQ ID NO:6, or SEQ ID NO:7; CDRH1 having an amino acid sequence of SEQ ID NO:8, SEQ ID NO:9, or SEQ ID NO:10; CDRH2 having an amino acid sequence of , SEQ ID NO:11, SEQ ID NO:12, SEQ ID NO:13, SEQ ID NO:14, SEQ ID NO:15, or SEQ ID NO:16; and/or a CDRH3 having an amino acid sequence of SEQ ID NO:17, SEQ ID NO:18, SEQ ID NO:19, SEQ ID NO:20, SEQ ID NO:21, or SEQ ID NO:22. In certain embodiments, combinations of the CDR sequences listed above may be used.

### II. ANTIGENS

An “antigen” is any substance that serves as a target for the receptors of an adaptive immune response, such as the T cell receptor, major histocompatibility complex class I and II, CD1d, B cell receptor or an antibody, or otherwise induces or increases an adverse immune response. An antigen may originate from within the body (“self,” “auto” or “endogenous”). An antigen may originate from outside the body (“non-self,” “foreign” or “exogenous”, or “allogeneic”), having entered, for example, by inhalation, ingestion, injection, or transplantation, and at times biochemically modified in the body. Foreign antigens include, but are not limited to, food antigens, animal antigens, plant antigens, environmental antigens, therapeutic agents, as well as antigens present in an allograft transplant. A conjugate may also be an assortment of one or more antigens, as with extracellular vesicles derived from B cells, dendritic cells, monocytes, or other antigen presenting cell, or serum plasma of a donor. In particular embodiments, the antigen is one in which a tolerogenic immune response is desired, whether that be anergy, deletion, or regulation.

An “epitope”, also known as antigenic determinant, is the segment of a macromolecule, e.g., a protein, which is recognized by the adaptive immune system, such as by antibodies, B cells, major histocompatibility complex molecules, CD1d molecules, T cells, or NKT cells. An epitope is that part or segment of a macromolecule capable of binding to an antibody or antigen-binding fragment thereof. In this context, the term “binding” in particular relates to a specific binding. According to some embodiments, the term “epitope” refers to the segment of protein or polypeptide that is recognized by the immune system.

In several embodiments, the antigen coupled to the LSECtin binding moiety or specific antibody or fragments thereof can be a protein or a peptide, e.g., the antigen may be a complete or partial therapeutic agent, a full-length transplant protein or peptide thereof, a full-length autoantigen or peptide thereof, a full-length allergen or peptide thereof, and/or a nucleic acid, or a mimetic of an antigen. The antigen can comprise an extracellular vesicle derived from B cells, dendritic cells, macrophages, monocytes, or other cell types, or from serum plasma, being transferred across major or minor antigen mismatches, such as from one MHC haplotype to another. In still additional embodiments, the antigen is housed in, integrated into or otherwise carried by, for example, cell fragments such as exosomes or extracellular vesicles or whole cells containing transplant or autoimmune antigens.

In certain aspects, antigens comprise, but are not limited to one or more (a), (b), (c) and (d), as follows (including combinations thereof): (a) Therapeutic agents that are proteins, peptides, lipids, saccharides, antibodies and antibody-like molecules, including antibody fragments and fusion proteins with antibodies and antibody fragments. These include, but are not limited to, human, non-human (such as mouse) and non-natural (e.g., engineered) proteins, antibodies, chimeric antibodies, humanized antibodies, camelid antibodies, and non-antibody binding scaffolds, such as protein frameworks including complementary determining regions such as fibronectins, knottins, anticalins, affibodies, 4-helix bundle proteins, ankyrin repeat proteins (e.g., DARPins), tetranectins, adnectins, A-domain proteins, lipocalins, immunity protein ImmE7, cytochrome b562, amyloid a-protein precursor inhibitor, cellulose binding domain from cellobiohydrolase Cel7A, carbohydrate binding module CBM4-2; RNA; DNA aptamers; and molecularly imprinted nanoparticles. (b) Human allograft transplantation antigens against which transplant recipients develop an unwanted immune response, including human or nonhuman cellular fragments such as exosomes or extracellular vesicles that contain specific transplantation antigens. (c) Self-antigens that cause an unwanted, autoimmune response. Although they are endogenous, for tolerance induction using the present compositions they could typically be synthesized exogenously (as opposed to being purified and concentrated from a source of origin). Alternatively, bifunctional linkers could associate with endogenous self-antigens in situ. (d) Foreign antigens, such as food, animal, plant and environmental antigens, against which a patient experiences an unwanted immune response. Those skilled in the art will appreciate that while a therapeutic protein can also be considered a foreign antigen due to its exogenous origin, for purposes of clarity in the description of the present disclosure such therapeutics are described as a separate group. Similarly, a plant or an animal antigen can be eaten and considered a food antigen, and an environmental antigen may originate from a plant. They are, however, all foreign antigens. In the interest of simplicity, no attempt will be made to describe, distinguish, and define all of such potentially overlapping groups, and listing or description of a particular antigen in a particular group does not preclude that member from potentially being considered as a member of another group, as those skilled in the art can appreciate the antigens that can be employed in the compositions of the disclosure, particularly in light of the detailed description and examples.

In several embodiments, the antigen can be a complete protein, a portion of a complete protein, a peptide, or the like (e.g., a mimetic or antigenic fragment), and can be derivatized (as discussed herein) for attachment to a linker moiety, can be a variant, and/or can contain conservative substitutions. As discussed above, a full-length antigen need not be used, rather, in several embodiments, an immunogenic fragment, or fragments, of an antigen are used. In some embodiments, multiple copies of an immunogenic fragment are used, e.g., the antigen portion of a composition comprises X1-X1-X1-X1 (an optional linker may be included between the X1 portions, in some embodiments). In some embodiments, multiple fragments of an antigen are used, e.g., X1, X2, X3, the fragments optionally being distinct regions of the antigen in some embodiments, while in other embodiments the regions can be at least partially overlapping. In still further embodiments, multiple fragments from multiple antigens are used, e.g., the antigen portion of a composition comprises X1, Y1, Z 1 .

In several embodiments, employing an antigen that is a therapeutic protein, peptide, antibody or antibody-like molecule, specific antigens can be selected from the following list, without limitation (Leader et al., Nat Rev Drug Discov 7:21-39, 2008, hereby incorporated by reference): Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor VIIa, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin [including recombinant human insulin (“rHu insulin”) and bovine insulin], Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Natalizumab, Octreotide, Ofatumumab, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, vW factor. The therapeutic protein can be obtained from natural sources (e.g., concentrated and purified) or synthesized, e.g., recombinantly, and includes antibody therapeutics that are typically IgG monoclonal or fragments or fusions.

In particular aspects the therapeutic protein, peptide, antibody or antibody-like molecules are Abciximab, Adalimumab, Agalsidase alfa, Agalsidase beta, Aldeslukin, Alglucosidase alfa, Factor VIII, Factor IX, Infliximab, Insulin (including rHu Insulin), L-asparaginase, Laronidase, Natalizumab, Octreotide, Phenylalanine ammonia-lyase (PAL), or Rasburicase (uricase) and generally IgG monoclonal antibodies in their varying formats.

Another particular group includes the hemostatic agents (Factor VIII and IX), Insulin (including rHu Insulin), and the non-human therapeutics uricase, PAL and asparaginase.

Unwanted immune responses in hematology and transplants include autoimmune aplastic anemia, transplant rejection (generally), and Graft vs. Host Disease (bone marrow transplant rejection). In the embodiments where the antigen is a human allograft transplantation antigen, specific sequences can be selected from: subunits of the various MHC class I and MHC class II haplotype proteins (for example, donor/recipient differences identified in tissue cross-matching), and single-amino-acid polymorphisms on minor blood group antigens including RhCE, Kell, Kidd, Duffy and Ss. Such compositions can be prepared individually for a given donor/recipient pair. In the embodiments where the antigen is a human allograft transplantation antigen, specific sequences may be present at purified molecular entities, or they may be contained in whole cells or cell fragments and extracellular vesicles, for examples from B cells, dendritic cells, macrophages, monocytes, or other cell types, or from blood serum or plasma, being transferred across major or minor mismatches, such as from one MHC haplotype to another.

In the embodiments where the antigen is a self-antigen or a derivative thereof, specific antigens (and the autoimmune disease with which they are associated) can be selected from, but not limited to:

In type 1 diabetes mellitus, several antigens have been identified and include, but are not limited to: insulin, proinsulin, preproinsulin, glutamic acid decarboxylase-65 (GAD-65 or glutamate decarboxylase 2), GAD-67, glucose-6 phosphatase 2 (IGRP or islet-specific glucose 6 phosphatase catalytic subunit related protein), insulinoma-associated protein 2 (IA-2), and insulinoma-associated protein 2β (IA-2β); other antigens include ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. It should be noted that insulin is an example of an antigen that can be characterized both as a self-antigen and a therapeutic protein antigen. For example, rHu Insulin and bovine insulin are therapeutic protein antigens (that are the subject of unwanted immune attack), whereas endogenous human insulin is a self-antigen (that is the subject of an unwanted immune attack). Because endogenous human insulin is not available to be employed in a pharmaceutical composition a recombinant form is employed in selected compositions of the disclosure.

Human insulin, including an exogenously obtained form useful in several embodiments, has the following sequence (UNIPROT P01308):

(SEQ ID NO: 23)
MALWMRLLPLIALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN.

GAD-65, including an exogenously obtained form useful in several embodiments, has the following sequence (UNIPROT Q05329):

(SEQ ID NO: 24)
MASPGSGFWSFGSEDGSGDSENPGTARAWCQVAQKFTGGIGNKLCALLYG
DAEKPAESGGSQPPRAAARKAACACDQKPCSCSKVDVNYAFLHATDLLPA
CDGERPTLAFLQDVMNILLQYVVKSFDRSTKVIDFHYPNELLQEYNWELA
DQPQNLEEILMHCQTTLKYAIKTGHPRYFNQLSTGLDMVGLAADWLTSTA
NTNMFTYEIAPVFVLLEYVTLKKMREIIGWPGGSGDGIFSPGGAISNMYA
MMIARFKMFPEVKEKGMAALPRLIAFTSEHSHFSLKKGAAALGIGTDSVI
LIKCDERGKMIPSDLERRILEAKQKGFVPFLVSATAGTTVYGAFDPLLAV
ADICKKYKIWMHVDAAWGGGLLMSRKHKWKLSGVERANSVTWNPHKMMGV
PLQCSALLVREEGLMQNCNQMHASYLFQQDKHYDLSYDTGDKALQCGRHV
DVFKLWLMWRAKGTTGFEAHVDKCLELAEYLYNIIKNREGYEMVFDGKPQ
HTNVCFWYIPPSLRTLEDNEERMSRLSKVAPVIKARMMEYGTTMVSYQPL
GDKVNFFRMVISNPAATHQDIDFLIEEIERLGQDL.

IGRP, including an exogenously obtained form useful in several embodiments, has the following sequence (UNIPROT QN9QR9):

(SEQ ID NO: 25)
MDFLHRNGVLIIQHLQKDYRAYYTFLNFMSNVGDPRNIFFIYFPLCFQFN
QTVGTKMIWVAVIGDWLNLIFKWILFGHRPYWWVQETQIYPNHSSPCLEQ
FPTTCETGPGSPSGHAMGASCVWYVMVTAALSHTVCGMDKFSITLHRLTW
SFLWSVFWLIQISVCISRVFIATHFPHQVILGVIGGMLVAEAFEHTPGIQ
TASLGTYLKTNLFLFLFAVGFYLLLRVLNIDLLWSVPIAKKWCANPDWIH
IDTTPFAGLVRNLGVLFGLGFAINSEMFLLSCRGGNNYTLSFRLLCALTS
LTILQLYHFLQIPTHEEHLFYVLSFCKSASIPLTVVAFIPYSVHMLMKQS
GKKSQ.

IA-2, including an exogenously obtained form useful in several embodiments, has the following sequence (NCBI Reference Sequence: XP 016860098.1):

(SEQ ID NO: 80)
MRRPRRPGGLGGSGGLRLLLCLLLLSSRPGGCSAVSAHGCLFDRRLCSHL
EVCIQDGLFGQCQVGVGQARPLLQVTSPVLQRLQGVIRQLMSQGLSWHDD
LTQYVISQEMERIPRLRPPEPRPRDRSGLAPKRPGPAGELLLQDIPTGSA
PAAQHRLPQPPVGKGGAGASSSLSPLQAELLPPLLEHLLLPPQPPHPSLS
YEPALLQPYLFHQFGSRDGSRVSEGSPGMVSVGPLPKAEAPALFSRTASK
GIFGDHPGHSYGDLPGPSPAQLFQDSGLLYLAQELPAPSRARVPRLPEQG
SSSRAEDSPEGYEKEGLGDRGEKPASPAVQPADAALQRLAAVLAGYGVEL
RQLTPEQLSTLLTLLQLLPKGAGRNPGGVVNVGADIKKTMEGPVEGRDTA
ELPARTSPMPGHPTASPTSSEVQQVPSPVSSEPPKAARPPVTPVLLEKKS
PLGQSQPTVAGQPSARPAAEEYGYIVTDQKPLSLAAGVKLLEILAEHVHM
SSGSFINISVVGPALTFRIRHNEQNLSLADVTQQAGLVKSELEAQTGLQI
LQTGVGQREEAAAVLPQTAHSTSPMRSVLLTLVALAGVAGLLVALAVALC
VRQHARQQDKERLAALGPEGAHGDTTFEYQDLCRQHMATKSLFNRAEGPP
EPSRVSSVSSQFSDAAQASPSSHSSTPSWCEEPAQANMDISTGHMILAYM
EDHLRNRDRLAKEWQALCAYQAEPNTCATAQGEGNIKKNRHPDFLPYDHA
RIKLKVESSPSRSDYINASPIIEHDPRMPAYIATQGPLSHTIADFWQMVW
ESGCTVIVMLTPLVEDGVKQCDRYWPDEGASLYHVYEVNLVSEHIWCEDF
LVRSFYLKNVQTQETRTLTQFHFLSWPAEGTPASTRPLLDFRRKVNKCYR
GRSCPIIVHCSDGAGRTGTYILIDMVINRMAKGVKEIDIAATLEHVRDQR
PGLVRSKDQFEFALTAVALEVNAILKALPQ.

In autoimmune diseases of the thyroid, including Hashimoto's thyroiditis and Graves' disease, antigens include, but are not limited to, thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor.

In Addison's Disease, antigens include, but are not limited to, 21-hydroxylase, 17α-hydroxylase, and P450 side chain cleavage enzyme (P450scc); other antigens include ACTH receptor, P450c21 and P450c17.

In premature ovarian failure, antigens include, but are not limited to, FSH receptor and α-enolase.

In autoimmune hypophysitis, or pituitary autoimmune disease, main antigens include, but are not limited to, pituitary gland-specific protein factor (PGSF) la and 2; another antigen is type 2 iodothyronine deiodinase.

In multiple sclerosis, antigens include, but are not limited to, myelin basic protein (“MBP”), myelin oligodendrocyte glycoprotein (“MOG”) and myelin proteolipid protein (“PLP”).

MBP, including an exogenously obtained form useful in several embodiments, has the following sequence (UNIPROT P02686):

(SEQ ID NO: 26)
MGNHAGKRELNAEKASTNSETNRGESEKKRNLGELSRTTSEDNEVFGEAD
ANQNNGTSSQDTAVTDSKRTADPKNAWQDAHPADPGSRPHLIRLFSRDAP
GREDNTFKDRPSESDELQTIQEDSAATSESLDVMASQKRPSQRHGSKYLA
TASTMDHARHGFLPRHRDTGILDSIGRFFGGDRGAPKRGSGKDSHHPART
AHYGSLPQKSHGRTQDENPVVHFFKNIVTPRTPPPSQGKGRGLSLSRFSW
GAEGQRPGFGYGGRASDYKSAHKGFKGVDAQGTLSKIFKLGGRDSRSGSP
MARR.

MOG, including an exogenously obtained form useful in several embodiments, has the following sequence (UNIPROT Q16653):

(SEQ ID NO: 27)
MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLIFLCLQYRLRGKLRAEIENLHRTFDPH
FLRVPCWKITLFVIVPVLGPLVALIICYNWLHRRLAGQFLEELRNPF.

PLP, including an exogenously obtained form useful in several embodiments, has the following sequence (UNIPROT P60201):

(SEQ ID NO: 28)
MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCHCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF.

Peptides/epitopes useful in several embodiments for treating multiple sclerosis include some or all of the following sequences, individually or in combination (including multiple repetitions of one or more of the following:

MBP13-32:
(SEQ ID NO: 29)
KYLATASTMDHARHGFLPRH;
MBP83-99:
(SEQ ID NO: 30)
ENPWHFFKNIVTPRTP;
MBP111-129:
(SEQ ID NO: 31)
LSRFSWGAEGQRPGFGYGG;
MBP146-170:
(SEQ ID NO: 32)
AQGTLSKIFKLGGRDSRSGSPMARR;
MOG1-20:
(SEQ ID NO: 33)
GQFRVIGPRHPIRALVGDEV;
MOG35-55:
(SEQ ID NO: 34)
MEVGWYRPPFSRWHLYRNGK;
PLP139-154:
(SEQ ID NO: 35)
HCLGKWLGHPDKFVGI,
MOG1-62:
(SEQ ID NO: 69)
GQFRVIGPRHPIRALVGDEVELPCRISPGKNATGMEVGWYRPPFSRVVHL
YRNGKDQDGDQA,
MBP76-136:
(SEQ ID NO: 70)
SHGRTQDENPVVHFFKNIVTPRTPPPSQGKGRGLSLSRFSWGAEGQRPGF
GYGGRASDYKSCG;
MBP1-50:
(SEQ ID NO: 71)
GCASQKRPSQRHGSKYLATASTMDHARHGFLPRHRDTGILDSIGRFFGGD
RG;
MBP131-170:
(SEQ ID NO: 72
ASDYKSAHKGFKGVDAQGTLSKIFKLGGRDSRSGSPMARRCG;
MBP102-136:
(SEQ ID NO: 74)
SQGKGRGLSLSRFSWGAEGQRPGFGYGGRASDYKSCG;
MOGI-27:
(SEQ ID NO: 75)
GQFRVIGPRHPIRALVGDEVELPCRIS;
and
MOG18-62:
(SEQ ID NO: 76)
DEVELPCRISPGKNATGMEVGWYRPPFSRVVHLYRNGKDQDGDQA.

In rheumatoid arthritis, antigens include, but are not limited to, collagen II, immunoglobulin binding protein, the fragment crystallizable region of immunoglobulin G, double-stranded DNA, and the natural and cirtullinated forms of proteins implicated in rheumatoid arthritis pathology, including fibrin/fibrinogen, vimentin, collagen I and II, and alpha-enolase.

In autoimmune gastritis, a non-limiting example of an antigen is H+,K+-ATPase.

In pernicious angemis, a non-limiting example of an antigen is intrinsic factor.

In celiac disease, antigens include, but are not limited to, tissue transglutaminase and the natural and deamidated forms of gluten or gluten-like proteins, such as alpha-, gamma-, and omega-gliadin, glutenin, hordein, secalin, and avenin. Those skilled in the art will appreciate, for example, that while the main antigen of celiac disease is alpha gliadin, alpha gliadin turns more immunogenic in the body through deamidation by tissue glutaminase converting alpha gliadin's glutamines to glutamic acid. Thus, while alpha gliadin is originally a foreign food antigen, once it has been modified in the body to become more immunogenic it can be characterized as a self-antigen. Peptides/epitopes useful in several embodiments for treating celiac disease include some or all of the following sequences, individually or in combination (including multiple repetitions of one or more of the following: DQ-2 related native gliadin: LQLQPFPQPQLPYPQPQLPYPQPQLPYPQPQPF (SEQ ID NO:42); DQ-2 related deamidated gliadin: LQLQPFPQPELPYPQPELPYPQPELPYPQPQPF (SEQ ID NO:43); DQ-8 related alpha-gliadin: QQYPSGQGSFQPSQQNPQ (SEQ ID NO:44), DQ-8 related omega-gliadin: QPFPQPEQPFPW (SEQ ID NO:45), an immunogenic fragment of gliadin: PQPELPY (SEQ ID NO:77), a deamidated fragment of gliadin: LQLQPFPQPQLPYPQPE (SEQ ID NO:78), and an additional fragment of gliadin: LQLQPFPQPQLPYPQPQ (SEQ ID NO:79).

In vitiligo, non-limiting examples of antigens are tyrosinase, and tyrosinase related protein 1 and 2.

MART1, Melanoma antigen recognized by T cells 1, Melan-A, including an exogenously obtained form useful in several embodiments , has the following sequence (UNIPROT Q16655):

(SEQ ID NO: 36)
MPREDAHFIYGYPKKGHGHSYTTAEEAAGIGILTVILGVLLLIGCWYCRR
RNGYRALMDKSLHVGTQCALTRRCPQEGFDHRDSKVSLQEKNCEPVVPNA
PPAYEKLSAEQSPPPYSP.

Tyrosinase, including an exogenously obtained form useful in several embodiments, has the following sequence (UNIPROT P14679):

(SEQ ID NO: 37)
MLLAVLYCLLWSFQTSAGHFPRACVSSKNLMEKECCPPWSGDRSPCGQLS
GRGSCQNILLSNAPLGPQFPFTGVDDRESWPSVFYNRTCQCSGNFMGFNC
GNCKFGFWGPNCTERRLLVRRNIFDLSAPEKDKFFAYLTLAKHTISSDYV
IPIGTYGQMKNGSTPMFNDINIYDLFVWMHYYVSMDALLGGSEIWRDIDF
AHEAPAFLPWHRLFLLRWEQEIQKLTGDENFTIPYWDWRDAEKCDICTDE
YMGGQHPTNPNLLSPASFFSSWQIVCSRLEEYNSHQSLCNGTPEGPLRRN
PGNHDKSRTPRLPSSADVEFCLSLTQYESGSMDKAANFSFRNTLEGFASP
LTGIADASQSSMHNALHIYMNGTMSQVQGSANDPIFLLHHAFVDSIFEQW
LRRHRPLQEVYPEANAPIGHNRESYMVPFIPLYRNGDFFISSKDLGYDYS
YLQDSDPDSFQDYIKSYLEQASRIWSWLLGAAMVGAVLTALLAGLVSLEC
RHKRKQLPEEKQPLLMEKEDYHSLYQSHL.

Melanocyte protein PMEL, gp100, including an exogenously obtained form useful in several embodiments, has the following sequence (UNIPROT P40967):

(SEQ ID NO: 38)
MDLVLKRCLLHLAVIGALLAVGATKVPRNQDWLGVSRQLRTKAWNRQLYP
EWTEAQRLDCWRGGQVSLKVSNDGPTLIGANASFSIALNFPGSQKVLPDG
QVIWVNNTIINGSQVWGGQPVYPQETDDACIFPDGGPCPSGSWSQKRSFV
YVWKTWGQYWQVLGGPVSGLSIGTGRAMLGTHTMEVTVYHRRGSRSYVPL
AHSSSAFTITDQVPFSVSVSQLRALDGGNKHFLRNQPLTFALQLHDPSGY
LAEADLSYTWDFGDSSGTLISRALVVTHTYLEPGPVTAQVVLQAAIPLTS
CGSSPVPGTTDGHRPTAEAPNTTAGQVPTTEVVGTTPGQAPTAEPSGTTS
VQVPTTEVISTAPVQMPTAESTGMTPEKVPVSEVMGTTLAEMSTPEATGM
TPAEVSIVVLSGTTAAQVTTTEWVETTARELPIPEPEGPDASSIMSTESI
TGSLGPLLDGTATLRLVKRQVPLDCVLYRYGSFSVTLDIVQGIESAEILQ
AVPSGEGDAFELTVSCQGGLPKEACMEISSPGCQPPAQRLCQPVLPSPAC
QLVLHQILKGGSGTYCLNVSLADTNSLAVVSTQLIMPGQEAGLGQVPLIV
GILLVEMAVVLASLIYRRRLMKQDFSVPQLPHSSSHWLRLPRIFCSCPIG
ENSPLLSGQQV.

In myasthenia gravis, a non-limiting example of an antigen is acetylcholine receptor.

In pemphigus vulgaris and variants, non-limiting examples of antigens are desmoglein 3, 1 and 4; other antigens include pemphaxin, desmocollins, plakoglobin, perplakin, desmoplakins, and acetylcholine receptor.

In bullous pemphigoid, non-limiting examples of antigens include BP180 and BP230; other antigens include plectin and laminin 5.

In dermatitis herpetiformis Duhring, non-limiting examples of antigens include, endomysium and tissue transglutaminase.

In epidermolysis bullosa acquisita, a non-limiting example of an antigen is collagen VII.

In systemic sclerosis, non-limiting examples of antigens include, but are not limited to, matrix metalloproteinase 1 and 3, the collagen-specific molecular chaperone heat-shock protein 47, fibrillin-1, and PDGF receptor; other antigens include Scl-70, Ul RNP, Tb/To, Ku, Jol, NAG-2, centromere proteins, topoisomerase I, nucleolar proteins, RNA polymerase I, II and III, PM-Slc, fibrillarin, and B23.

In mixed connective tissue disease, a non-limiting example of an antigen is Ul snRNP.

In Sjogren's syndrome, non-limiting examples of antigens are nuclear antigens SS-A and SS-B; other antigens include fodrin, poly(ADP-ribose) polymerase and topoisomerase, muscarinic receptors, and the Fc-gamma receptor IIIb.

In systemic lupus erythematosus, non-limiting examples of antigens include nuclear proteins including the “Smith antigen,” SS-A, high mobility group box 1 (HMGB1), nucleosomes, histone proteins and double-stranded DNA (against which auto-antibodies are made in the disease process).

In Goodpasture's syndrome, non-limiting example of antigens include glomerular basement membrane proteins including collagen IV.

In rheumatic heart disease, a non-limiting example of an antigen is cardiac myosin.

In autoimmune polyendocrine syndrome type 1, non-limiting example of antigens include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In neuromyelitis optica, a non-limiting example of an antigen is AQP4.

Aquaporin-4, including an exogenously obtained form useful in several embodiments , has the following sequence (UNIPROT P55087):

(SEQ ID NO: 39)
MSDRPTARRWGKCGPLCTRENIMVAFKGVWTQAFWKAVTAEFLAMLIFVL
LSLGSTINWGGTEKPLPVDMVLISLCFGLSIATMVQCFGHISGGHINPAV
TVAMVCTRKISIAKSVFYIAAQCLGAIIGAGILYLVTPPSVVGGLGVTMV
HGNLTAGHGLLVELIITFQLVFTIFASCDSKRTDVTGSIALAIGFSVAIG
HLFAINYTGASMNPARSFGPAVIMGNWENHWIYWVGPIIGAVLAGGLYEY
VFCPDVEFKRRFKEAFSKAAQQTKGSYMEVEDNRSQVETDDLILKPGVVH
VIDVDRGEEKKGKDQSGEVESSV.

In uveitis, non-limiting examples of antigens include Retinal S-antigen or “S-arrestin” and interphotoreceptor retinold binding protein (IRBP) or retinol-binding protein 3.

S-arrestin, including an exogenously obtained form useful in several embodiments , has the following sequence (UNIPROT P10523):

(SEQ ID NO: 40)
MAASGKTSKSEPNHVIFKKISRDKSVTIYLGNRDYIDHVSQVQPVDGVVL
VDPDLVKGKKVYVTLTCAFRYGQEDIDVIGLTERRDLYESRVQVYPPVGA
ASTPTKLQESLLKKLGSNTYPFLLTFPDYLPCSVMLQPAPQDSGKSCGVD
FEVKAFATDSTDAEEDKIPKKSSVRLLIRKVQHAPLEMGPQPRAEAAWQF
FMSDKPLHLAVSLNKEIYFHGEPIPVTVTVTNNTEKTVKKIKAFVEQVAN
VVLYSSDYYVKPVAMEEAQEKVPPNSTLTKTLTLLPLLANNRERRGIALD
GKIKHEDTNLASSTIIKEGIDRTVLGILVSYQIKVKLTVSGFLGELTSSE
VATEVPFRLMHPQPEDPAKESYQDANLVFEEFARHNLKDAGEAEEGKRDK
NDVDE.

IRBP, including an exogenously obtained form useful in several embodiments, has the following sequence (UNIPROT P10745):

(SEQ ID NO: 41)
MMREWVLLMSVLLCGLAGPTHLFQPSLVLDMAKVLLDNYCFPENLLGMQE
AIQQAIKSHEILSISDPQTLASVLTAGVQSSLNDPRLVISYEPSTPEPPP
QVPALTSLSEEELLAWLQRGLRHEVLEGNVGYLRVDSVPGQEVLSMMGEF
LVAHVWGNLMGTSALVLDLRHCTGGQVSGIPYIISYLHPGNTILHVDTIY
NRPSNTTTEIWTLPQVLGERYGADKDVVVLTSSQTRGVAEDIAHILKQMR
RAIVVGERTGGGALDLRKLRIGESDFFFTVPVSRSLGPLGGGSQTWEGSG
VLPCVGTPAEQALEKALAILTLRSALPGVVHCLQEVLKDYYTLVDRVPTL
LQHLASMDFSTVVSEEDLVTKLNAGLQAASEDPRLLVRAIGPTETPSWPA
PDAAAEDSPGVAPELPEDEAIRQALVDSVFQVSVLPGNVGYLRFDSFADA
SVLGVLAPYVLRQVWEPLQDTEHLIMDLRHNPGGPSSAVPLLLSYFQGPE
AGPVHLFTTYDRRTNITQEHFSHMELPGPRYSTQRGVYLLTSHRTATAAE
EFAFLMQSLGWATLVGEITAGNLLHTRTVPLLDTPEGSLALTVPVLTFID
NHGEAWLGGGVVPDAIVLAEEALDKAQEVLEFHQSLGALVEGTGHLLEAH
YARPEVVGQTSALLRAKLAQGAYRTAVDLESLASQLTADLQEVSGDHRLL
VFHSPGELVVEEAPPPPPAVPSPEELTYLIEALFKTEVLPGQLGYLRFDA
MAELETVKAVGPQLVRLVWQQLVDTAALVIDLRYNPGSYSTAIPLLCSYF
FEAEPRQHLYSVFDRATSKVTEVWTLPQVAGQRYGSHKDLYILMSHTSGS
AAEAFAHTMQDLQRATVIGEPTAGGALSVGIYQVGSSPLYASMPTQMAMS
ATTGKAWDLAGVEPDITVPMSEALSIAQDIVALRAKVPTVLQTAGKLVAD
NYASAELGAKMATKLSGLQSRYSRVTSEVALAEILGADLQMLSGDPHLKA
AHIPENAKDRIPGIVPMQIPSPEVFEELIKFSFHTNVLEDNIGYLRFDMF
GDGELLTQVSRLLVEHIWKKIMHTDAMIIDMRFNIGGPTSSIPILCSYFF
DEGPPVLLDKIYSRPDDSVSELWTHAQVVGERYGSKKSMVILTSSVTAGT
AEEFTYIMKRLGRALVIGEVTSGGCQPPQTYHVDDTNLYLTIPTARSVGA
SDGSSWEGVGVTPHVVVPAEEALARAKEMLQHNQLRVKRSPGLQDHL.

In the embodiments where the antigen is a foreign antigen against which an unwanted immune response can be developed, such as food antigens, specific antigens include, but are not limited to: Peanut antigen(s): conarachin (Ara h 1), allergen II (Ara h 2), arachis agglutinin, conglutin (Ara h 6); conarachin, for example has the sequence identified as UNIPROT Q6PSU6; Apple antigen: 31 kda major allergen/disease resistance protein homolog (Mal d 2), lipid transfer protein precursor (Mal d 3), major allergen Mal d 1.03D (Mal d 1); Milk antigen: a-lactalbumin (ALA), lactotransferrin; from kiwi: actinidin (Act c 1, Act d 1), phytocystatin, thaumatin-like protein (Act d 2), kiwellin (Act d 5); egg white antigen: ovomucoid, ovalbumin, ovotransferrin, and lysozyme; egg yolk antigen: livetin, apovitillin, and vosvetin; mustard antigen: 2S albumin (Sin a 1), 11S globulin (Sin a 2), lipid transfer protein (Sin a 3), profilin (Sin a 4); celery antigen: profilin (Api g 4), high molecular weight glycoprotein (Api g 5); shrimp antigen: Pen a 1 allergen (Pen a 1), allergen Pen m 2 (Pen m 2), tropomyosin fast isoform; wheat or other cereal antigen: gliadin, high molecular weight glutenin, low molecular weight glutenin, alpha-, gamma- and omega-gliadin, hordein, secalin and/or avenin; strawberry antigen: major strawberry allergy Fra a 1-E (Fra a 1); and banana antigen: profilin (Mus xp 1).

Peptides/epitopes useful, in several embodiments, for treating Celiac Disease include some or all of the following sequences, individually or in combination:

DQ-2 relevant, Alpha-gliadin “33-mer” native:
(SEQ ID NO: 42)
LQLQPFPQPQLPYPQPQLPYPQPQLPYPQPQPF;
DQ-2 relevant, Alpha-gliadin “33-mer” deamidated:
(SEQ ID NO: 43)
LQLQPFPQPELPYPQPELPYPQPELPYPQPQPF;
DQ-8 relevant, Alpha-gliadin:
(SEQ ID NO: 44)
QQYPSGQGSFQPSQQNPQ;
DQ-8 relevant, Omega-gliadin (wheat, U5UA46):
(SEQ ID NO: 45)
QPFPQPEQPFPW.

In the embodiments where the antigen is a foreign antigen against which an unwanted immune response is developed, such as to animal, plant and environmental antigens, specific antigens can, include, but are not limited to, for example: cat, mouse, dog, horse, bee, dust, tree and goldenrod, including the following proteins or peptides derived from: (a) weeds, (including ragweed allergens amb a 1, 2, 3, 5, and 6, and Amb t 5; pigweed Che a 2 and 5; and other weed allergens Par j 1, 2, and 3, and Par o 1); (b) grass (including major allergens Cyn d 1, 7, and 12; Dac g 1, 2, and 5; Hol I 1.01203; Lol p 1, 2, 3, 5, and 11; Mer a 1; Pha a 1; Poa p 1 and 5); (c) pollen from ragweed and other weeds (including curly dock, lambs quarters, pigweed, plantain, sheep sorrel, and sagebrush), grass (including Bermuda, Johnson, Kentucky, Orchard, Sweet vernal, and Timothy grass), and trees (including catalpa, elm, hickory, olive, pecan, sycamore, and walnut); (d) dust (including major allergens from species Dermatophagoides pteronyssinus, such as Der p 1, 2, 3, 4, 5, 6, 7, 8, 9, 10, 11, 14, 15, 18, 20, 21, and 23; from species Dermatophagoides farina, such as Der f 1, 2, 3, 6, 7, 10, 11, 13, 14, 15, 16, 18, 22, and 24; from species Blomia tropicalis such as Blo t 1, 2, 3, 4, 5, 6, 10, 11, 12, 13, 19, and 21; also allergens Eur m 2 from Euroglyphus maynei, Tyr p 13 from Tyrophagus putrescentiae, and allergens Bla g 1, 2, and 4; Per a 1, 3, and 7 from cockroach); (e) pets (including cats, dogs, rodents, and farm animals; major cat allergens include Fel d 1 through 8, cat IgA, BLa g 2, and cat albumin; major dog allergens include Can f 1 through 6, and dog albumin); (f) bee stings, including major allergens Api m 1 through 12; and (g) fungus, including allergens derived from, species of Aspergillus and Penicillium, as well as the species Alternaria alternata, Davidiella tassiana, and Trichophyton rubrum.

In several embodiments, with respect to the formula [A-B-X], Xis an antibody, antibody fragment or ligand that specifically binds a circulating protein or peptide or antibody, which circulating protein or peptide or antibody gives rise to transplant rejection, immune response against a therapeutic agent, autoimmune disease, and/or allergy (or other unwanted immune reaction).

In several embodiments, with respect to the formula [A-B-X], X binds an endogenous circulating protein or peptide or antibody.

In several embodiments, with respect to the formula [A-B-X], X is a fluorophore such as Alexa Fluor 405, Alexa Fluor 488, Alexa Fluor 555, Alexa Fluor 594, Alexa Fluor 647, Alexa Fluor 700, AmCyan, allophycocyanin (APC), APC/Alexa Fluor 750, APC/Cy5.5, APC/Cy7, BD Horizon V450, BD Horizon V500, BD Horizon BB515, BD Horizon BUV395, BD Horizon BUV4956, BD Horizon BUV737, Brilliant Violet 421, Brilliant Violet 510, Brilliant Violet 570, Brilliant Violet 605, Brilliant Violet 650, Brilliant Violet 711, Brilliant Violet 785, Cascade Blue, Cascade Yellow, CFP, CFSE, Cy3, Cy5, DAPI, DRAQS, DRAQ7, DsRed-Express, dTomato, eBFP, eCFP, eFluor 450, eFluor 565NC, eFluor 605NC, eFluor 650NC, eFluor 700NC, FITC, Flash Phalloidin RED 594, Flash Phalloidin NIR 647, GFP, Helix NP NIR, Hoechst 33258, mCherry, MitoSpy Green FM, MitoSpy Orange CMTMRos, mPlum, NADH, Pacific Blue, Pacific Orange, phycoerythrin (PE), PE-CF594, PE/Cy5, PE/Cy5.5, PE/Cy7, PE/Dazzle 594, PE/Texas Red-X, PerCP, PerCP/Cy5.5, PerCP=eFluor 710, Propidium Iodide, Qdot 525, Qdot 545, Qdot 565, Qdot 585, Qdot 605, Qdot 625, Qdot 655, Qdot 705, Qdot 800, Riboflavin, Tag-it Violet, TO-PRO-3, YFP, Zombie Aqua, Zombie Greem, Zombie NIR, Zombie Red, Zombie UV, Zombie Violet, Zombie Yellow, ZsGreen.

In several embodiments, with respect to the formula [A-B-X], X is an extracellular vesicle. To isolate extracellular vesicles, antigen-presenting cells (monocytes, B cells, or dendritic cells) can be isolated from mice using , for example, magnetic bead isolation. Cells are cultured for 3 days in RPMI (4% exosome-free fetal bovine serum, 1% penicillin/streptomycin) at density of 1 million cells/mL. After 3 days, supernatant is isolated and centrifuged at 300×g for 5 minutes at 4 C to pellet cells. Supernatant is isolated and centrifuged at 2000×g for 5 minutes at 4 C. Supernatant is isolated and centrifuged at 10,000×g for 30 minutes at 4 C. Supernatant is taken and centrifuged at 100,000×g for 70 minutes to pellet extracellular vesicles. Pellet is washed in PBS and centrifuged again at 100,000×g for 70 minutes. Pellet is resuspended in PBS and concentration is measured using bicinchoninic assay (BCA). Pellet may be aliquoted and stored at −20C. Alternatively, exosomes may be isolated from a cell line culture, or directly from serum isolated from whole blood.

According to several embodiments, a patient can be tested to identify an antigen against which an unwanted immune response has developed, and a protein, peptide or the like can be developed based on that antigen and incorporated as X in a composition according to embodiments of the present disclosure.

### III. LINKERS

Linkers, such as amino acid or peptidimimetic sequences may be inserted between the LSECtin binding moiety and antigen. Linkers may have one or more properties that include a flexible conformation, an inability to form an ordered secondary structure or a hydrophobic or charged character which could promote or interact with either domain. Examples of amino acids t_{yp}ically found in flexible protein regions may include Gly, Asn, and Ser. Other near neutral amino acids, such as Thr and Ala, may also be used in the linker sequence. The length of the linker sequence may vary without significantly affecting the function or activity of the fusion protein (see, U.S. Pat. No. 6,087,329). In a particular aspect, a LBM and antigen are joined by a peptide sequence having from about 1, 2, 3, 4, 5, 6, 7, 8, 9, 10, 11, 12, 13, 14, 15, 16, 17, 18, 19, 20, 21, 22, 23, 24, to 25 amino acid residues. Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Linkers further include a linear carbon chain, such as CN (where N=1−100 carbon atoms, e.g., C₁, C₂, C₃, C₄, C₅, C₆, C₇, C₈). In some embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some embodiments, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Further, the linker may be maleimidocaproyl (mc).

In certain embodiments a linker is a bifunctional linker and includes reagents for molecular conjugation reactions to provide structural stability or assistance in protein-cell, protein-cell fragment, protein-exosome, protein-extracellular vesicle, protein-protein, protein-peptide, protein-polymer, polymer-small molecule, peptide/protein-small molecule interactions, immobilization for assays or purification, as well as various peptide-nucleic acid and nucleic-nucleic acid conjugations, among many others. Typically, bifunctional linkers contain functional groups, such as primary amines, sulfhydryls, acids, alcohols and bromides. Specifically maleimide (sulfhydryl reactive) and succinimidyl ester (NHS) or isothiocyanate (ITC) groups that react with amines may find used in the current embodiments.

In certain aspects, a bifunctional linker can be used as a spacer between an LSECtin binding moiety and an antigen. Linking groups can include, but are not limited to, ester, carbonate, carbamate, imine (hydrazine), amide, maleimide, succinimidyl, vinylsulfone, conjugated C═C double bond, epoxy, aldehyde, ketone, silane or siloxane functionalities. Without limitation to theory, several embodiments also encompasses cleavable linkers used in chemical biology classified according to their cleavage conditions by, for example, enzymes, nucleophilic/basic reagents, photo-irradiation, electrophilic/acidic reagents, organometallic and metal reagents, or oxidizing reagents.

In certain aspects a LSECtin binding moiety can be linked to extracellular vesicles and the like. A LSECtin binding moiety can be chemically conjugated to extracellular vesicles, cell fragments, or cells. LSECtin binding moieties may also be linked to extracellular vesicles, cell fragments, or cells via non-covalent mechanisms (Armstrong et al., Therapeutics. ACS Nano, 2017).

A bi-functional molecule may be produced by recombinant expressed or chemical conjugation, such that on one side it is the LSECtin binding moiety, and on the other binds to a protein or molecule on the extracellular vesicle, cell fragment, or cell, (e.g., a tetraspanin). Cells may be genetically engineered to express a LSECtin binding moiety with a membrane insertion sequence, such that once expressed, LSECtin binding moiety is inserted into the membrane, and cells and all derivatives thereof will have a LSECtin binding moiety inserted into the membrane. A LSECtin binding moiety may be recombinantly expressed such that it has a hydrophobic membrane insertion region that may be inserted into extracellular vesicles, cell fragments, or cells in vitro. Extracellular vesicles, cell fragments, and cells may be permeabilized, such as by electroporation, to allow for a LSECtin binding moiety to be inserted into the membrane.

### IV. RELATED METHODS OF USE

Various embodiments of the compositions of the present disclosure find use in a variety of applications including, but not limited to, detection of LSECtin protein such as by flow cytometry, western blot, and immunohistochemistry, and treatment of transplant rejection, immune response against a therapeutic agent, autoimmune disease, and food allergy.

In several embodiments, the compositions of the disclosure are used to modulate, particularly down-regulate, antigen-specific undesirable immune response.

In several embodiments, compositions disclosed herein are useful to bind and clear from the circulation specific undesired proteins, including antibodies endogenously generated in a patient (i.e., not exogenous antibodies administered to a patient), peptides and the like, which cause autoimmunity and associated pathologies, allergy, inflammatory immune responses, and anaphylaxis.

In several embodiments, antigens are targeted to the liver for presentation via liver sinusoidal endothelial cells (LSECs) to specifically down-regulate the immune system or for clearance of unwanted circulating proteins.

Several embodiments of the present disclosure provide compositions and methods to treat unwanted immune response to self-antigens and foreign antigens, including but not limited to: a foreign transplant antigen against which transplant recipients develop an unwanted immune response (e.g., transplant rejection), a foreign antigen to which patients develop an unwanted immune (e.g., allergic or hypersensitivity) response, a therapeutic agent to which patients develop an unwanted immune response (e.g., hypersensitivity and/or reduced therapeutic activity), a self-antigen to which patients develop an unwanted immune response (e.g., autoimmune disease).

Autoimmune disease states that can be treated using the methods and compositions provided herein include, but are not limited to: Acute Disseminated Encephalomyelitis (ADEM); Acute interstitial allergic nephritis (drug allergies); Acute necrotizing hemorrhagic leukoencephalitis; Addison's Disease; Alopecia areata; Alopecia universalis; Ankylosing Spondylitis; Arthritis, juvenile; Arthritis, psoriatic; Arthritis, rheumatoid; Atopic Dermatitis; Autoimmune aplastic anemia; Autoimmune gastritis; Autoimmune hepatitis; Autoimmune hypophysitis; Autoimmune oophoritis; Autoimmune orchitis; Autoimmune polyendocrine syndrome type 1; Autoimmune polyendocrine syndrome type 2; Autoimmune thyroiditis; Behcet's disease; Bronchiolitis obliterans; Bullous pemphigoid; Celiac disease; Churg-Strauss syndrome; Chronic inflammatory demyelinating polyneuropathy; Cicatricial pemphigoid; Crohn's disease; Coxsackie myocarditis; Dermatitis herpetiformis Duhring; Diabetes mellitus (Type 1); Erythema nodosum; Epidermolysis bullosa acquisita, Giant cell arteritis (temporal arteritis); Giant cell myocarditis; Goodpasture's syndrome; Graves' disease; Guillain-Bane syndrome; Hashimoto's encephalitis; Hashimoto's thyroiditis; IgG4-related sclerosing disease; Lambert-Eaton syndrome; Mixed connective tissue disease; Mucha-Habermann disease; Multiple sclerosis; Myasthenia gravis; Optic neuritis; Neuromyelitis optica; Pemphigus vulgaris and variants; Pernicious angemis; Pituitary autoimmune disease; Polymyositis; Postpericardiotomy syndrome; Premature ovarian failure; Primary Biliary Cirrhosis; Primary sclerosing cholangitis; Psoriasis; Rheumatic heart disease; Sjogren's syndrome; Systemic lupus erythematosus; Systemic sclerosis; Ulcerative colitis; Undifferentiated connective tissue disease (UCTD); Uveitis; Vitiligo; and Wegener's granulomatosis.

A particular group of autoimmune disease states that can be treated using the methods and compositions provided herein include, but are not limited to: Acute necrotizing hemorrhagic leukoencephalitis; Addison's Disease; Arthritis, psoriatic; Arthritis, rheumatoid; Autoimmune aplastic anemia; Autoimmune hypophysitis; Autoimmune gastritis; Autoimmune polyendocrine syndrome type 1; Bullous pemphigoid; Celiac disease; Coxsackie myocarditis; Dermatitis herpetiformis Duhring; Diabetes mellitus (Type 1); Epidermolysis bullosa acquisita; Giant cell myocarditis; Goodpasture's syndrome; Graves' disease; Hashimoto's thyroiditis; Mixed connective tissue disease; Multiple sclerosis; Myasthenia gravis; Neuromyelitis optica; Pernicious angemis; Pemphigus vulgaris and variants; Pituitary autoimmune disease; Premature ovarian failure; Rheumatic heart disease; Systemic sclerosis; Sjogren's syndrome; Systemic lupus erythematosus; and Vitiligo.

In the embodiments employing an antigen against which an unwanted immune response is developed, such as food antigens, treatment can be provided for reactions against, for example: peanut, apple, milk, egg whites, egg yolks, mustard, celery, shrimp, wheat (and other cereals), strawberry and banana.

According to several embodiments, a patient can be tested to identify a foreign antigen against which an unwanted immune response has developed, and a composition of the disclosure can be developed based on that antigen.

### V. TESTING

In certain embodiments, specificity of the compositions provided for herein in binding to liver and sinusoidal endothelial cells (LSECs) in vivo can be established. This can be accomplished, for example, by employing a marker (such as the fluorescent marker Alexa Fluor 647) in a composition of the disclosure. The composition is administered to suitable experimental subjects. Controls, e.g., irrelevant Fab or vehicle (saline) are administered to other group(s) of subjects. The composition and controls are allowed to circulate for a period of 10 minutes to 5 hours, after which the spleens and livers of the subjects are harvested and measured for fluorescence. The specific cells in which fluorescence is found can be subsequently identified. Alternatively, experimental subjects may be imaged in real time using an in vivo imaging system. Compositions of the disclosure, when tested in this manner, show higher levels of concentration in the antigen-presenting cells of the liver as compared with irrelevant Fab or vehicle.

Humoral immune response can be tested by administering a composition of the disclosure incorporating a known antigen, such as OVA (a gold standard antigen in immunological testing), as compared with the administration of the antigen alone or antigen conjugated to an irrelevant Fab, and measuring the levels of resulting antibodies. In several embodiments, compositions of the disclosure when tested in this manner, in several embodiments, show very low (e.g., background) levels of antibody formation responsive to their administration and the administration of vehicle, with significantly higher levels of antibody formation responsive to administration of the antigen or antigen conjugated to irrelevant Fab.

Disease-focused experimental models are well known to those skilled in the art and include the NOD (or non-obese diabetic) mouse model of autoimmunity and tolerance and the EAE (experimental autoimmune encephalomyelitis) model for the human inflammatory demyelinating disease, multiple sclerosis. In particular, immunization with myelin oligodendrocyte glycoprotein (MOG) or immunogenic peptides derived from MOG, emulsified in complete Freud's adjuvant (CFA) leads to immune-mediated demyelination and symptoms mimicking those of multiple sclerosis. Fabs may be chemically conjugated or recombinantly expressed with MOG or MOG peptides to assess prevention and treatment of EAE.

To measure transplantation tolerance, extracellular vesicles will be isolated as described above from BALB/c mice, which express the H2-K^{d} haplotype of major histocompatibility molecules (MFIC). Fab will be conjugated to extracellular vesicles as described above. Fab-EV will be injected into C57B1/6J mice, which express the H2-K^{b} haplotype of MHC, serving as a complete MHC mismatch. Tail skin from Balb/c mice will be transplanted onto the flank of C57B1/6J mice that previously received Fab-EV. Grafts will be checked daily for signs of necrosis or rejection. Grafts will be considered rejected if they are over 20% necrotic or if they fall off. Grafts will be considered accepted if they remain 60 days after transplantation.

### VI. ADMINISTRATION

The compositions of the disclosure are administered at a therapeutically effective dosage, e.g., a dosage sufficient to provide treatment for the disease states previously described. Administration of the compounds of the disclosure can be via any of the accepted modes of administration for agents that serve similar utilities.

Depending on the embodiment, generally in mice, the doses in mice are from the about 2.5 μg to 200 ng/gram body weight. Generally, an individual human dose is from about 0.01 to 2.0 mg/kg of body weight, about 0.1 to 1.5 mg/kg of body weight, or about 0.3 to 1.0 mg/kg of body weight, depending on the embodiment, or any dose between those listed, including the endpoints. Treatment can be administered for a single day or a period of days, and can be repeated at intervals of several days, one or several weeks, or one or several months. Administration can be as a single dose (e.g., as a bolus) or as an initial bolus followed by continuous infusion of the remaining portion of a complete dose over time, e.g., 1 to 7 days. The amount of active compound administered may, depending on the embodiment, be dependent on any or all of the following: the subject and disease state being treated, the severity of the affliction, the manner and schedule of administration and the judgment of the prescribing physician. It will also be appreciated that amounts administered may depend upon the molecular weight of the antigen, antibody, antibody fragment or ligand as well as the size of the linker, which may vary from embodiment to embodiment.

Depending on the embodiment, the compositions of the disclosure can be administered either alone or in combination with other pharmaceutically acceptable excipients. While all typical routes of administration are contemplated, several embodiments provide for liquid dosage forms suitable for injection. The formulations may include a conventional pharmaceutical carrier or excipient and a composition of the disclosure or a pharmaceutically acceptable salt thereof. In addition, these compositions can include other medicinal agents, pharmaceutical agents, carriers, and the like, including, but not limited to the therapeutic protein, peptide, antibody or antibody-like molecule corresponding to the antigen (X) employed in the composition of the disclosure, and other active agents that can act as immune-modulating agents and more specifically can have inhibitory effects on B-cells, including anti-folates, immune suppressants, cytostatics, mitotic inhibitors, and anti-metabolites, or combinations thereof.

Generally, depending on the intended mode of administration, the pharmaceutically acceptable composition will contain about 0.1% to 95%, or about 0.5% to 50%, by weight of a composition of the disclosure, the remainder being suitable pharmaceutical excipients, carriers, etc. Dosage forms or compositions containing active ingredient in the range of 0.005% to 95% with the balance made up from non-toxic carrier can be prepared.

Liquid pharmaceutically administrable compositions can, for example, be prepared by dissolving, dispersing, etc. an active composition of the disclosure (e.g., a lyophilized powder) and optional pharmaceutical adjuvants in a carrier, such as, for example, water (water for injection), saline, aqueous dextrose, glycerol, glycols, ethanol or the like, to thereby form a solution or suspension. If desired, the pharmaceutical composition to be administered can also contain minor amounts of nontoxic auxiliary substances such as wetting agents, emulsifying agents, stabilizing agents, solubilizing agents, pH buffering agents and the like, for example, sodium acetate, sodium citrate, cyclodextrine derivatives, sorbitan monolaurate, triethanolamine acetate and triethanolamine oleate, etc., osmolytes, amino acids, sugars and carbohydrates, proteins and polymers, salts, surfactants, chelators and antioxidants, preservatives, and specific ligands. Actual methods of preparing such dosage forms are known, or will be apparent, to those skilled in this art. The composition or formulation to be administered will, in any event, contain a quantity of the active compound in an amount effective to treat the symptoms of the subject being treated.

### VII. EXAMPLES

The following examples as well as the figures are included to demonstrate non-limiting embodiments of the inventions disclosed herein. It should be appreciated by those of skill in the art that the techniques disclosed in the examples or figures represent non-limiting techniques and those of skill in the art should, in light of the present disclosure, appreciate that many changes can be made in the specific embodiments which are disclosed and still obtain a like or similar result without departing from the spirit and scope of the invention.

### EXAMPLE 1

### Design and Expression of Target LSECtin Forms

The full-length sequence of murine LSECtin was ordered from Genscript. To generate LSECtin protein lacking the transmembrane and cytosolic domain, PCR amplification was conducted to create three variants of LSECtin corresponding to amino acids 54-294, 100-294, and 155-294. At the N-terminus of the vector, the secretion signal from Laminin-II was added to allow for expression of secreted soluble protein. Following the laminin subunit gamma II secretion signal (MPALWLGCCLCFSLLLPAARNLAGT (SEQ ID NO:46)), the sequence for SNAP tag (NEB) was added to enable site-specific biotinylation. At the C-terminus of the vector, a thrombin-cleavable site followed by a (His)6 tag was included to enable protein purification on a Ni-NTA column. The entire sequence was cloned into the pHEK293 Ultra expression vector (Takara). The plasmid was transfected into HEK suspension cells seeded at 1 million cells per mL. After 6-8 days of culture, supernatant was harvested, passed through a 0.22 μM filter, and purified on an HisTrap column on the Akta pure 25 M system (GE Healthcare). Protein was washed with 30 mM imidazole in 25 mM Tris-Hcl, 300 mM NaCl, and eluted with 500 mM imidazole in 25 mM Tris-HCl 300 mM NaCl. SNAP-LSECtin was dialyzed overnight against 5 L of 25 mM Tris-HCl , 150 mM NaCl, 10 mM CaCl₂. For long term storage of SNAP-LSECtin, 10% glycerol was added and protein was stored at −80° C.

### Example 2

### Selection of Phage Library

The phage library used was kindly provided by Anthony Kossiakoff at the University of Chicago. The library consists of humanized Fab based on the anti-HER2 antibody 4D5. Of the six complementarity determining regions (CDR), CDR-L1 and CDR-L2 from the light chain are constant, CDR-L3, CDR-H1, and CDR-H2 have limited diversity, and CDR-H3 is completely randomized. The actual diversity of the library is 3×10¹⁰, which can cover a broad range of targets.

### Example 3

### Phage Screening

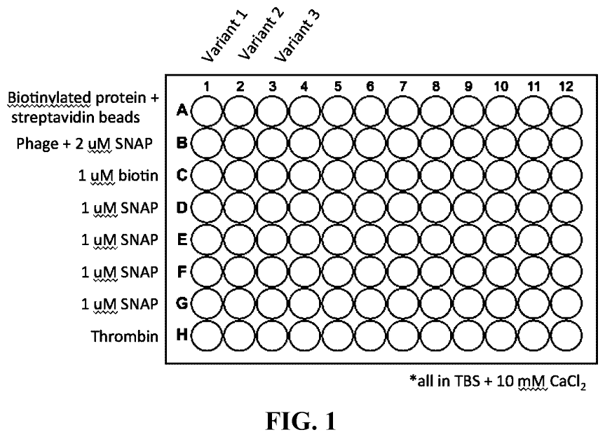

1 μM of each of the three SNAP-LSECtin variants was biotinylated as per the manufacturer's protocol (SNAP-biotin, NEB). For the first round, 5 x 10¹² phages were used for panning against 1μM of all three variants of LSECtin in parallel bound to 200 μL magnetic streptavidin beads (Promega) in PBST-BSA (PBS, 0.05% Tween, 0.5% bovine serum albumin) for 1 hour at room temperature. Magnetic beads were washed 4 times in PB ST-BSA. Phages were eluted from beads with thrombin. Eluted phages were incubated with 5 mL of XL1 Blue with M13-K07 helper phage overnight at 37° C. to propagate the phage. Cells were pelleted and supernatant containing phage was kept. PEG-NaCl (20% PEG 8k, 2.5 M NaCl) was added to supernatant at equal volumes to precipitate phage. Phage was centrifuged and suspended in PBST-BSA for the next round of panning. For subsequent panning, KingFisher plates were used with the KingFisher device in a plate setup as demonstrated in FIG. 1. The first row included streptavidin beads (Dynabeads) with 300 nM, 150 nM, 75 nM, or 20 nM biotinylated LSECtin variants, corresponding to the second, third, fourth, and fifth rounds of display. The next well row contained phage and 2μM biotinylated SNAP protein, to remove potential SNAP binders. The following row contained 1μM biotin to saturate biotin sites on the streptavidin beads. The following 4 rows contained 1μM SNAP to wash. The last row contained thrombin, to elute phage. All steps contained TBS +10 mM CaC12. Eluted phage was incubated with XL1, helper phage, and ampicillin overnight at 37° C.

### Example 4

### Phage Sequencing

After 5 rounds of panning on each of the three LSECtin variants, XL1 with phage was plated on LB-agar plates supplemented with ampicillin. Single clones were grown overnight at 37° C. in 96 deep well plates in 400 uL 2XYT supplemented with 100 ug/mL ampicillin and M13-K07 helper phage. Three plates corresponding to panning on the three variants of LSECtin were sent to the DNA Sequencing Core Facility at the University of Chicago.

### Example 5

### Hit Fab Expression

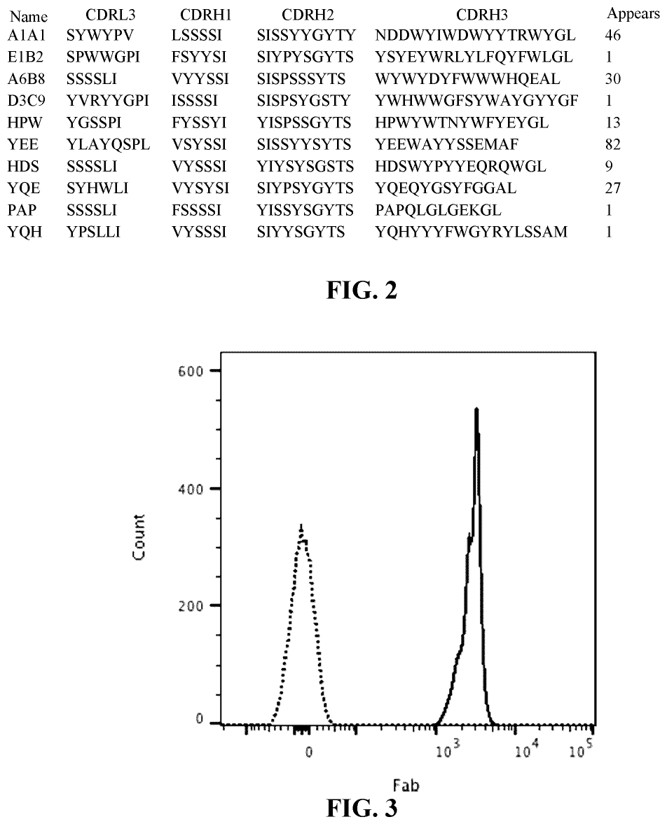

There were six sequences on which all clones converged of the three plates sequenced (FIG. 2). Using the forward primer 5′-CGCAACTTATTACTGTCAGC-3′ (SEQ ID NO:47) and reverse complement 5′-AGACGGTGACCAGGGTTCC-3′ (SEQ ID NO:48), Fab light and heavy chain sequences were PCR amplified with SuperFi PCR (Invitrogen) and run on a gel. The sequence of interest was cut and gel purified. pSFV4 plasmid was cut using (NdeI cut site) and used to ligate Fab PCR fragments with InFusion Cloning Kit. Ligated product was transfected into Stellar competent cells and plated on LB-agar plates supplemented with ampicillin. Single clones were sequenced to confirm proper ligation. Fab-pSFV4 plasmids were transfected into BL21-DE3 in overnight cultures of 5 mL in 2XYT supplemented with ampicillin. E. coli was grown up in a 1L volume in a 4L flask of 2XYT supplemented with 100 μg/mL ampicillin until an optical density of 0.6-0.8 was achieved. Protein expression was then induced by 1 mM Isopropyl β-D-1-thiogalactopyranoside for four hours. Cells were harvested by centrifugation and stored at −20° C. The following day cell pellets from were suspended in 30 mL phosphate buffered saline supplemented with protease inhibitors and benzonase and sonicated on ice. Lysate was spun down at 10,000 RPM to remove debris, and supernatant was incubated with Protein G resin provided by Anthony Kossiakoff for 1 hour at 4° C. Resin was spun down at 1000 RPM for 2 minutes, and subsequently washed with phosphate buffered saline. Resin was washed with 30 column volumes of phosphate buffered saline with 500 mM NaCl. Fabs were eluted with 100 mM glycine, pH 2.6. For assays involving protein analysis, Fabs were neutralized with 1 M Tris-HCl, pH 8, and dialyzed overnight against 5 L phosphate buffered saline. For assays involving in vivo use of Fabs, once Fabs were eluted with 100 mM glycine pH 2.6 they underwent cation exchange chromatography to remove endotoxin. In brief, Fabs were applied to HiTrap column (GE Healthcare) and washed with 50 mM sodium acetate buffer, pH 4.5. Fabs were eluted with a gradient up to 600 mM NaCl in 50 mM sodium acetate buffer, pH 4.5. Fractions were pooled and dialyzed against 5 L of PBS. After dialysis, samples were concentrated with 10,000 molecular weight cutoff ultracentrifugal filters (Amicon).

### Example 6

### Flow Cytometry for Validation of Fab Binding to LSEC tin

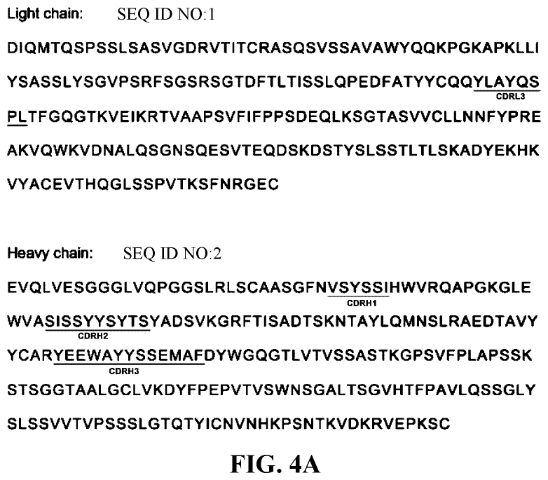

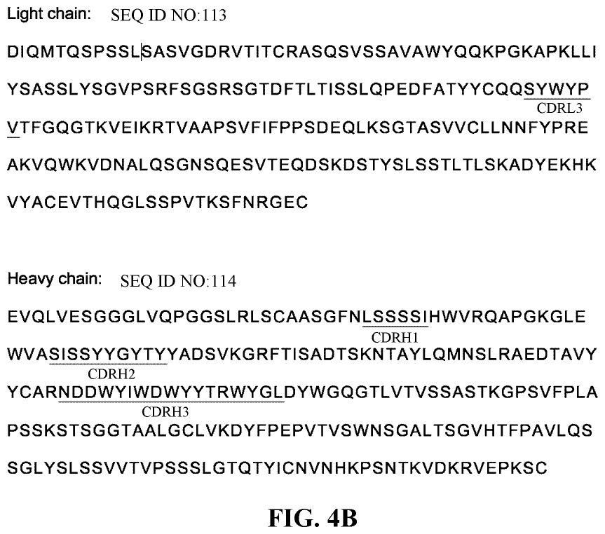

1μM SNAP-LSECtin was incubated with 1μM SNAP-biotin (NEB) and 1 mM DTT for 30 minutes at room temperature. 100 μL of biotinylated SNAP-LSECtin was incubated with 100 μL Avidin polystyrene beads (Spherotech) in 800 μL of Tris-HCl, pH 5.5 on a rotator for 1 hour at room temperature. 30 μL of beads +SNAP-LSECtin was added to 5 mL polystyrene tubes (Falcon), washed with 2 mL TBS +2% BSA +10 mM CaCl₂ and spun down at 2000 RPM for 5 minutes. Supernatant was discarded, and Fabs were added at a final concentration of 5 μg/mL for 15 minutes at room temperature. Samples were washed with 2 mL TBS +2% BSA +10 mM CaCl₂ and spun down at 2000 RPM for 5 minutes. Anti-human F(ab)2-Alexa Fluor 594 secondary antibody (Jackson Immunoresearch) was added at a final concentration of 1 μg/mL for 15 minutes at room temperature and washed. Samples were run on the Fortessa (BD) for flow cytometric analysis. Of all six Fabs expressed, the Fab with the CDRH3 ‘YEEWAYYSSEMAF’ (SEQ ID NO:17), referred to as YEE (FIG. 3; irrelevant Fab with the dashed line, YEE with the solid line) appeared to exhibit enhanced binding to LSECtin. The full sequence of YEE with CDRs highlighted may be found in FIG. 4A and for A1A1 in FIG. 4B.

### Example 7

### In Vitro Validation of Fab Binding to LSECS

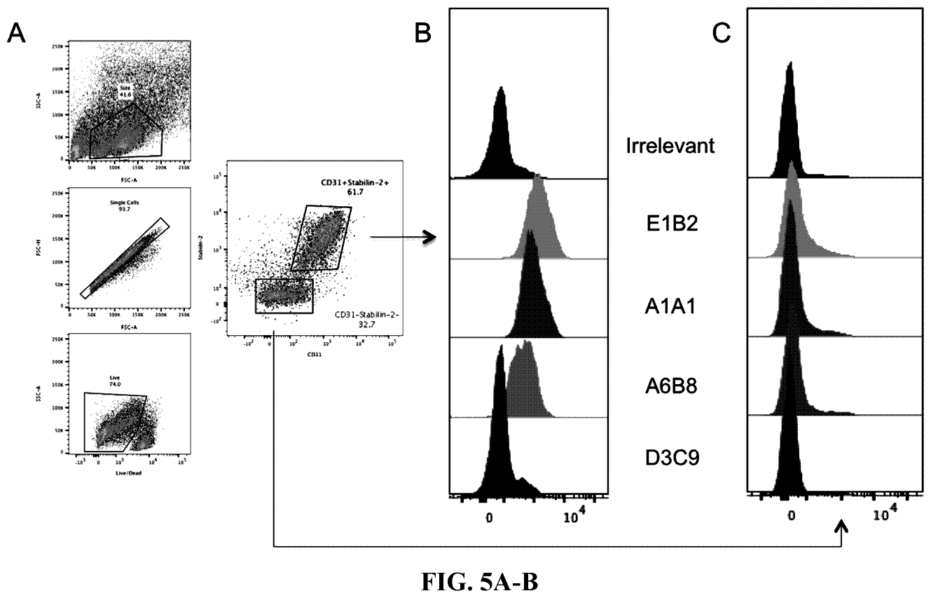

To confirm binding of Fabs to LSECs in vitro, LSECs were isolated from mouse livers as previously described (Meyer et al., Exp. Cell Res. 349:291-301, 2016). Briefly, mice were sacrificed and catheter was inserted in the inferior vena cava. The liver was perfused with 25 mL of calcium-free HBSS supplemented with 12.5 μmol EGTA, 125 units heparin, 62.5 μL 40% glucose, 625 μmol HEPES, and 1% penicillin/streptomycin. To digest the liver, it was then perfused under a heating lamp with IMDM supplemented with GlutaMax, 25 mg Collagenase IV (Worthington) and 2μg DNAse I (Sigma). Liver was excised and cells were immediately removed in a petri dish and passed through a 70 μM cell strainer. Cells were centrifuged at 68×g to remove pelleted hepatocytes. Supernatant was centrifuged at 600×g to pellet all remaining cells. Cells were suspended in 10 mL of DM EM. A two-step Percoll gradient was created by placing 20 mL of 50% Percoll as the bottom layer, 20 mL of 25% Percoll as top layer, and layering 10 mL of cell suspension on top. Cells were immediately spun at 1350×g with no brake. The resulting layer of cells between the two gradients was taken and washed with PBS. Cells were first stained with Live/Dead Viability Dye (Invitrogen) and Fc block (BD). Cells were washed with PBS +2% FBS. Cells were stained for CD31, Stabilin II, and CD45 and with 5 μg/mL Fab for 30 minutes at 4° C. Cells were washed in PBS +2% FBS and stained with 1:400 dilution of anti-Fab conjugated to APC for 15 minutes at 4° C. Cells were washed and fixed in 2% paraformaldehyde for 15 minutes at 4° C. Cells were washed and analyzed by flow cytometry (FIG. 5).

### Example 8

### Evaluation in Animal Models with the Model Antigens

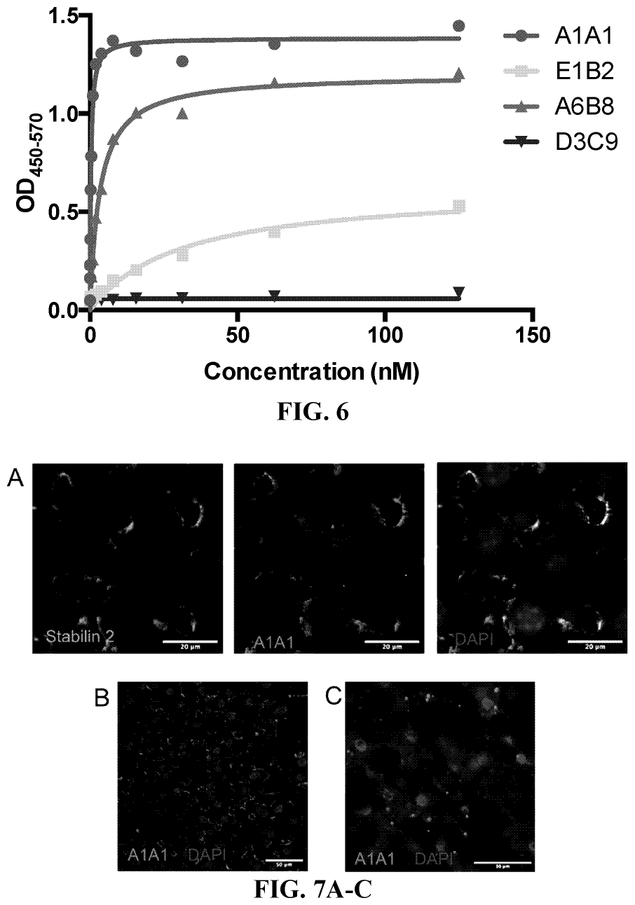

To measure the ability to induce antigen-specific T cell tolerance, derivatives of a model antigen, ovalbumin, were recombinantly expressed on the C terminus of the heavy chain of the YEE Fab separated by a Gly3Ser linker. Specifically, the CD8 epitope of ovalbumin recognized by the OTI TCR,'SIINFEKL' (SEQ ID NO:115), or the CD4 epitope of ovalbumin recognized by the OTII TCR ‘ISQAVHAAHAEINEAGREVVG’ (also referred to by shorthand as “ISQ”, SEQ ID NO:116) were expressed. These were flanked by amino acids involved in the natural antigen cleavage site. 500,000 OTI or OTII cells were injected into the tail vein of C57/BL6 mice. One day or 7 days later, mice were injected with 40 picomoles of YEE-SIINFEKL(‘YEEWAYYS SEMAF’-‘SIINFEKL’ (SEQ ID NO:117), YEE-ISQ (‘YEEWAYYSSEMAF’-‘ISQAVHAAHAEINEAGREVVM’SEQ ID NO:118), free SIINFEKL (SEQ ID NO:115) peptide, free ISQ peptide, or saline. On day 13, mice were challenged with either 10 μg of ovalbumin with 50 ng of lipopolysaccharide in the footpad or saline (naïve control). Mice were sacrificed at day 18 and lymph nodes and spleen were analyzed, as seen in FIG. 6A-B, for numbers of OTI and OTII cells, ability to synthesize effector molecules (e.g., interferon gamma) upon restimulation with antigen, and markers of exhaustion or tolerance.

### Example 9

### Fusion of Fab to Autoimmune Antigen

Anti-LSECtin Fab may be recombinantly expressed with, or chemically conjugated to, an autoimmune antigen or derivative thereof for induction of tolerance to said autoimmune antigen. For induction of tolerance to an immunodominant epitope of myelin oligodendrocyte protein, a major immune target of multiple sclerosis, MOG₂₀₋₆₀ may be recombinantly expressed with the Fab, for example on the C terminus of the heavy chain of the Fab, but also may be expressed on other locations in the Fab that do not disrupt binding to LSECtin. The antigen expressed will include the natural cleavage sites in the protein, as was done above for immunodominant epitopes of ovalbumin, such that the immunogenic epitope will be processed and presented as would occur naturally. This may be done for any autoimmune antigen.

### Example 10

### Coupling of Fab to Extracellular Vesicle

Anti-LSECtin Fab may be conjugated to extracellular vesicles for induction of tolerance to those antigens on the extracellular vesicle, for example for induction of tolerance to major histocompatibility complexes. This may be done by chemical conjugation or by a variety of other methods as described above. An example of a conjugation strategy is that Fabs may be recombinantly expressed to include a cysteine, which has a free thiol. Sulfosuccinimidyl 4-(N-maleimidomethyl)cyclohexane-1 -carboxylate (Sulfo-SMCC) may be used to react its maleimide on the one hand with the free thiol on the Fab, and the NHS-ester on the other hand with free amines on the extracellular vesicles.

### Example 11

### Enzyme-Linked Immunosorbent Assay for Fab Binding to LSECtin

Nunc MaxiSorp plates were coated overnight at 4° C. with 10 μg/mL LSECtin in sodium bicarbonate buffer. Plates were washed 3X in PBST with an ELISA plate washer. Plates were blocked for 2 hours in PBS +2% BSA at room temperature. Plates were washed 3× with an ELISA plate washer. Fabs were added at concentrations from 30 pM to 125 nM in PBS +2% BSA for 2 hours at room temperature. Plates were washed 5× in PBST with an ELISA plate washer. Horseradish peroxidase-conjugated anti-F(ab)2 IgG (Jackson Immunoresearch) was added at 1:5000 dilution in PBS +2% BSA for 1 hour at room temperature. Plates were washed 5X with an ELISA plate washer. TMB substrate was added and quenched with 10% sulfuric acid. Plates were read with a spectrophotometer at 450 nm wavelength and 570 reference wavelength. Data is shown in FIG. 6. These data demonstrate that, in accordance with several embodiments disclosed herein, Fab constructs generated and described herein have the ability to bind to LSECtin as evidenced by the increased optical density vs. concentration.

### Example 12

### Immunofluorescence of Anti-ISECtin Fab Binding to Liver Sections

Mice were perfused with Hank's Buffered Salt Solution followed by zinc fixative to fix the liver. Livers were fixed overnight in zinc fixative, transferred to a 10% sucrose solution for 24 hours at 4° C., and then to a 30% sucrose solution for 24 hours at 4° C. Livers were flash frozen and cryosectioned. Sections were stained with A1A1 anti-LSECtin Fab or irrelevant Fab control overnight, and rat anti-mouse Stabilin 2 (MBL International) at 4° C. in 0.5% casein in TBST. Sections were washed and stained with anti-human F(ab)2 Alexa Fluor 594 (Jackson Immunoresearch) and anti-rat Alexa Fluor 488 secondary antibodies for 1 hour at room temperature in 0.5% casein in TBST. Sections were mounted with ProLong Gold Antifade Mountant with DAPI (Life Technologies) and imaged on an Olympus confocal microscope. Data are shown in FIG. 7. (A) Mouse sections stained with stabilin 2 and A1A1 and imaged with 60×magnification. (B) Mouse sections stained with A1A1 and imaged with 20×magnification. (C) Monkey sections stained with A1A1and imaged with 60×magnification.

### Example 13

### Uptake Analysis of Anti-LSECtin Fab

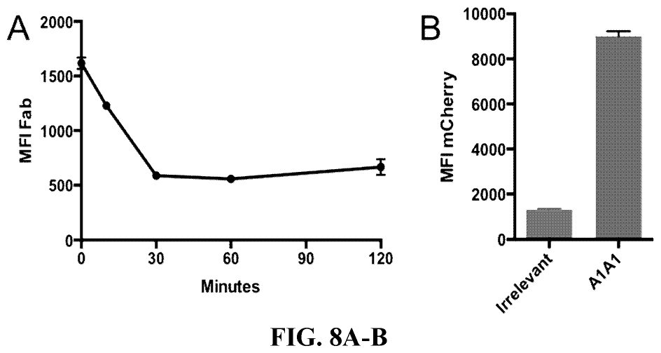

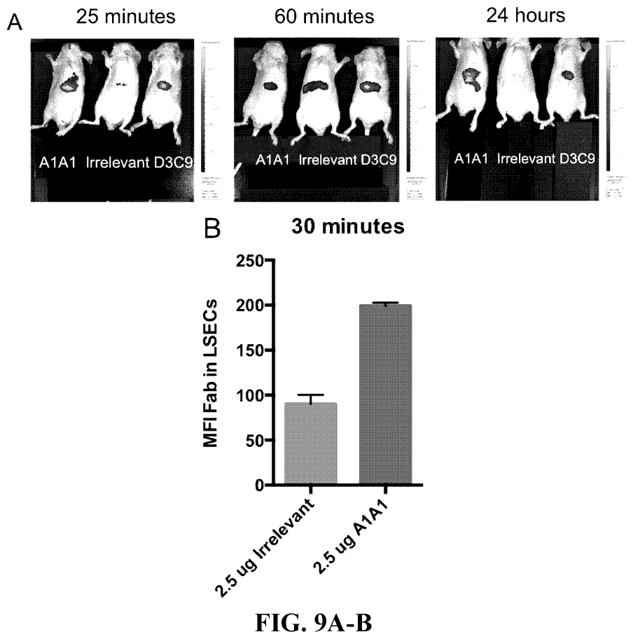

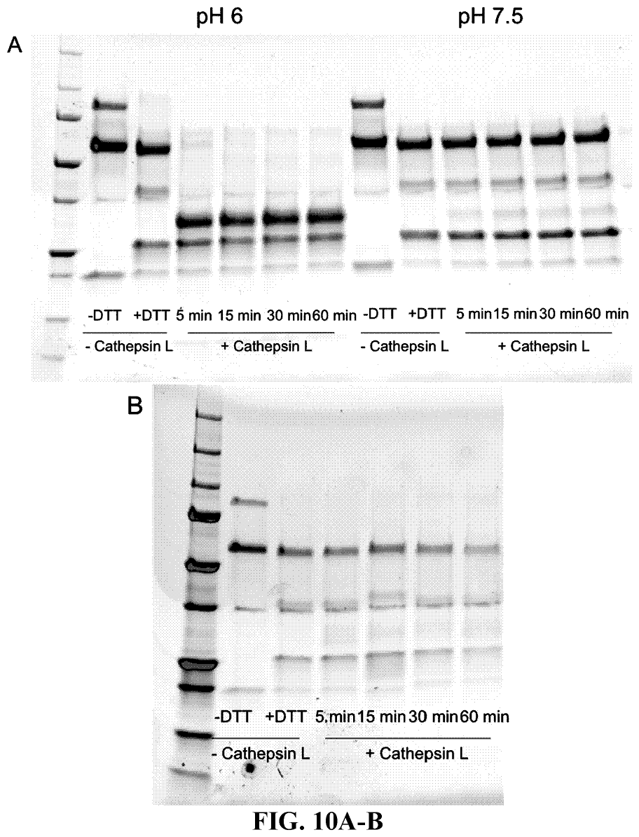

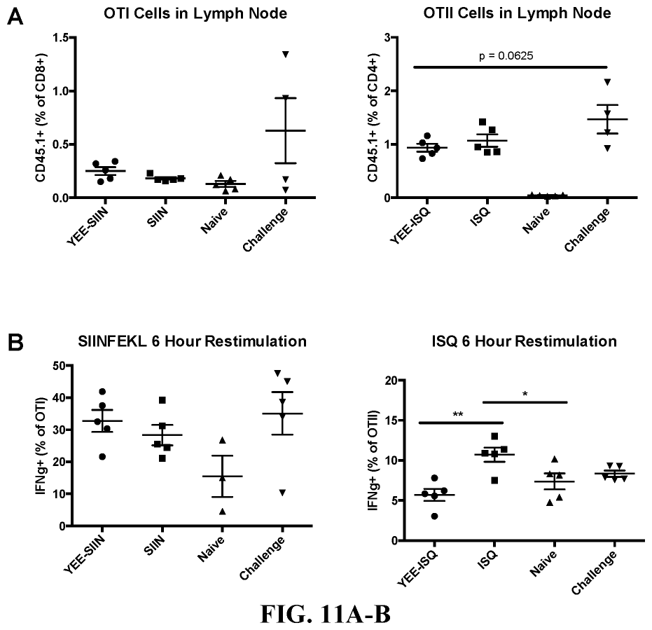

LSECs were isolated from mice as described above and sorted based on the expression of CD31 and Stabilin 2 and lack of CD45 and F4/80. (FIG. 8A) Fab was recombinantly expressed with mCherry on the heavy chain. Fab-mCherry was added to LSECs at 4° C. for 20 minutes and washed to remove excess Fab. LSECs were incubated at 37° C. to allow for endocytosis and subsequently stained with an anti-Fab antibody. (FIG. 8B) LSECs were incubated with A1A1-mCherry or irrelevant Fab-mCherry 2 hours at 37° C. and the flourescence intensity of mCherry was measured by flow cytometry. The enhanced signal detected and shown in FIG. 8B indicate that Fab are taken up (e.g., endocytosed) by LSECs, which further indicates that the tolerogenic compositions disclosed herein would be internalized by the LSECs (through binding to LSECtin), leading to processing of the antigen by the immune system to be recognized as self—thus inducing tolerance to the antigen.

### Example 14

### Biodistribution of Anti-LSECtin Fab In Vivo

To measure the ability to localize to LSECs in vivo, anti-LSECtin Fabs A1A1 and D3C9, as a Fab of irrelevant specificity, were conjugated to DY-800 (Dyomics), a near infrared fluorescent small molecule. 25 jug of Fab-800 were injected into nude mice. Fluorescence was measured in nude mice after 25 minutes, 60 minutes and 24 hours (IVIS, Perkin Elmer)(A). FIG. 8A shows the imaging data that demonstrates that Fab A1A1yields highly specific localization to the liver, in contrast to a Fab of irrelevant specificity. Similarly, Fab D3C9 also demonstrated localization to the liver in vivo. (B) To measure uptake specifically by LSECs, Fabs were conjugated to the fluorescent dye DY-649 (Dyomics). 2.5 μg of Fab-649 were injected into mice, and mice were sacrificed 30 minutes after injection. LSECs were isolated as in Example 7 and analyzed by flow cytometry for the presence of Fab, as indicated by mean fluorescence intensity of the 649 nm signal. These data show an increased localization of Fab A1A1 (as a non-limiting example) to the LSECs after in vivo administration. As above, these data support the localization (e.g., to the liver) of tolerogenic compositions comprising an LSECtin binding agent coupled to an antigen to which tolerance is desired.

### Example 15

### Design of Cathepsin Cleaveable Linkers Between Fab and Payload

Cathepsin cleaveable linkers were designed between the Fab and payload to allow for separation in acidic compartments and enhanced degradation and antigen presentation. RNAseq data reveals that the most prevalent cathepsins in LSECs are cathepsin L and cathepsin B (Ding et al., Mol. Cell Proteomics 15:3190-202, 2016). Potential sequences were obtained from Sudo et al. who performed mass spectrometry on peptide isolates after incubation of cells with the respective cathepsins and identified predicted cathepsin specificities (Sudo et al., J Control Release 255:1-11, 2017). Sequences were chosen based on abundance and adherence to predicted cathepsin specificities. Fabs were designed to have a Gly4Ser linker, cathepsin cleaveable sequence, and payload (OVA or mCherry). To determine if Fab constructs with payload separated by cathepsin cleaveable linkers were cleaved by cathepsins, proteins were incubated at 0.2 μg/mL with 1:100 mouse cathepsin L in pH 6 or pH 7.5 for varying time points at 37° C.

Sequences of cathepsin cleaveable linkers

CtsL1
(SEQ ID NO: 97)
YGYTHLSTGDLLR
CtsB
(SEQ ID NO: 98)
LPPPIGGAGPPLGLPK
CtsL5
(SEQ ID NO: 99)
LFIGGLSFET

### Example 16

### Design of Endosomal Escape-Fab Fusions to Enhance Antigen Presentation

In order to enhance class I MHC presentation, Fabs were designed to include endosomal escape peptides. After Fab internalization and delivery to cytosolic compartments, endosomal escape peptides would enable Fab-payload release from the endosome, transfer to cytoplasm, and degradation by the proteasome. This would lead to enhanced presentation on class I MHC. Various versions of endosomal escape peptides, utilizing cathepsin cleaveable linkers, were designed to facilitate payload escape from the endosome. INF7, a variant of the HA2 fusogenic peptide derived from influenza hemagglutinin, was chosen as it has been widely demonstrated to enhance endosomal escape (Plank et al., Journal of Biological Chem, 1994). A second fusogenic peptide derived from syncytin 1, a human fusogenic protein involved in placental development, was chosen for its translational potential (Sudo et al., J Control Release 255:1-11, 2017). Reports have demonstrated that although endosomal escape peptides may burst the endosome, cargo may be trapped in the membrane. To overcome this, cathepsin cleaveable linkers were added along with escape peptides so that if the escape peptide is bound to membrane, cathepsins will be able to cleave payload and it may be released into the cytoplasm. In addition to cathepsin cleaveable linkers, various linkers such as SPDP may be used to join Fab, payload, and endosomal escape peptide such that the linkers would be reduced in an endosome or lysosome and the payload released from the escape peptide.

| | INF7 | Syncytin 1 |
|---|---|---|
| Fab-X-Cts-EEP | GGGGSGGGGSYGYTHLSTGD | GGGGSGGGGSLFIGGLSFETPFVIGAGVLGAL |
| | LLRGLFEAIEGFIENGWEGMID | GTGIGGI (SEQ ID NO: 102) |
| | GWYG (SEQ ID NO: 101) | |
| Fab-EEP-Cts-X | GGGGSGGGGSGAAAGLFEAIE | GGGGSGGGGSGAAAPFVIGAGVLGALGTGI |
| | GFIENGWEGMIDGWYGYTHL | GGLSFE (SEQ ID NO: 104) |
| | STGDLLR (SEQ ID NO: 103) | |
| Fab-EEP-X | GGGGSGGGGSAAAGLFEAIEG | GGGGSGGGGSGAAAPFVIGAGVLGALGTGI |
| | FIENGWEGMIDGWYG (SEQ ID | GGI (SEQ ID NO: 106) |
| | NO: 105) | |
| Fab-X-EEP | GGGSGGGSGLFEAIEGFIENG | GGGSGGGSPFVIGAGVLGALGTGIGGI (SEQ |
| | WEGMIDGWYG (SEQ ID | ID NO: 108) |
| | NO: 107) | |
| Fab-link-X-EEP | GGGSC (SEQ ID NO: 119)-linker- | GGGSC (SEQ ID NO: 119)-linker-X- |
| | X- | GGGSGGGSPFVIGAGVLGALGTGIGGI (SEQ |
| | GGGSGGGSGLFEAIEGFIENG | ID NO: 110) |
| | WEGMIDGWYG (SEQ ID | |
| | NO: 109) | |
| Fab-X-link-EEP | X-GGGSC (SEQ ID NO: 119)- | X-GGGSC (SEQ ID NO: 119)-linker- |
| | linker- | GGGSGGGSPFVIGAGVLGALGTGIGGI (SEQ |
| | GGGSGGGSGLFEAIEGFIENG | ID NO: 112) |
| | WEGMIDGWYG (SEQ ID | |
| | NO: 111) | |

Where X is payload, Cts is cathepsin cleaveable linker, EEP is endosomal escape peptide, and linker is chemical linker that may be reduced in an acidic compartment.

### Example 17

### Assessing Endosomal Escape Variants

To assess ability of Fabs with endosomal escape peptides to escape into the cytoplasm, Fabs will be recombinantly expressed with the aforementioned sequences (Example 12) and mCherry as payload. LSECs will be isolated as described above and cultured on glass coverslips. LSECs will be incubated with Fab-EEP-mCherry variants for 20 minutes at 4° C. LSECs will then be incubated at 37° C. for 1 hour, washed, fixed in 2% paraformaldehyde, stained with markers of early and late endosomal compartments, and imaged using a confocal microscope. An escape peptide will be considered effective if mCherry is no longer seen colocalizing with endosomal markers but rather diffuse in the cytoplasm. In parallel, LSECs will be isolated, pulsed with Fab-mCherry endosomal escape variants, and live imaged with a lattice lightsheet microscope to observe live escape from endosomal compartments.

### Example 18

### Presentation on Class I And Class II MHC In Vitro

All aforementioned variants with cathepsin cleaveable linkers and endosomal escape peptides will be expressed recombinantly with ovalbumin as payload. LSECs will be isolated and cultured. LSECs will be pulsed with 100 μg/mL Fab-OVA cathepsin and endosomal escape variants for 12-16 hours, and stained with an anti-H2-Kb-SIINFEKL antibody which recognizes CD8 immunodominant epitope SIINFEKL presented on class I MHC. In parallel, OTI and OTII T cells, which recognize the CD8 and CD4 epitopes of ovalbumin presented on class I MHC and class II MHC, respectively, will be added to LSECs pulsed with Fab-OVA variants and assessed for proliferation and markers of activation.

### Example 19

### Tolerance to A Model Antigen

To determine tolerance in a model system in vivo, the Fab-OVA variants that showed best antigen presentation in above experiment will be expressed. OTI and DTII cells will be adoptively transferred to mice, followed by intravenous injections of Fab-OVA variants. Mice will be challenged with subcutaneous injection of ovalbumin and lipopolysaccharide in the footpad. 4-7 days later the mice will be sacrificed and T cell responses measured for markers of anergy and tolerance.

Although the foregoing has been described in some detail by way of illustrations and examples for purposes of clarity and understanding, it will be understood by those of skill in the art that modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the embodiments of the invention(s).

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering an LSECtin-binding protein” include “instructing the administration of an LSECtin-binding protein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ including but not limited to,' or the like.

The indefinite article “a” or “an” does not exclude a plurality. The term “about” as used herein to, for example, define the values and ranges of molecular weights means that the indicated values and/or range limits can vary within 120%, e.g., within 110%. The use of “about” before a number includes the number itself. For example, “about 5” provides express support for “5”. Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

1. A composition for induction of antigen-specific tolerance, the composition comprising:
a binding moiety that binds to human Liver Sinusoidal Endothelial Cell C-Type Lectin (LSECtin) comprising a heavy chain complementarity determining region (CDRH) comprising an amino acid sequence of SISSYY (SEQ ID NO:100); and an antigen to which tolerance is desired,
wherein the antigen to which tolerance is desired comprises one or more antigens or one or more fragments of said one or more antigens,
wherein the antigen to which tolerance is desired is covalently coupled to the LSECtin-binding moiety or joined to the LSECtin-binding moiety via a linker,
wherein a subject exposed to the antigen alone reacts to the antigen alone with an unwanted immune response, and
wherein a subject exposed to the composition has a reduced immune response to a subsequent exposure to the antigen.

2. The composition of claim 1, wherein the LSECtin-binding moiety is an LSECtin-specific antibody or a fragment of an LSECtin-specific antibody.

3. The composition of claim 1, wherein the LSECtin-binding moiety is fragment of an LSECtin-specific antibody.

4. The composition of claim 3, wherein the LSECtin-binding moiety further comprises an additional CDRH comprising an amino acid sequence of SSI.

5. The composition of claim 4, wherein the CDRH comprises an amino acid sequence of SISSYYX₃YTX₄ (SEQ ID NO:68) and the additional CDRH comprises an amino acid sequence of X₁SX₂SSI (SEQ ID NO:67).

6. The composition of claim 5, wherein the LSECtin-binding moiety further comprises at least a third CDRH and a light chain complementarity determining region (CDRL), wherein the CDRH comprises an amino acid sequence of SISSYYGYTY (SEQ ID NO:59) and the additional CDRH comprises an amino acid sequence of LSSSSI (SEQ ID NO:55).

7. (canceled)

8. The composition of claim 5, wherein the LSECtin-binding moiety further comprises a light chain complementarity determining region (CDRL) having an amino acid sequence of SYWYPV (SEQ ID NO:51) and at least a third CDRH having an amino acid sequence of NDDWYIWDWYYTRWYGL (SEQ ID NO:63).

9. (canceled)

10. The composition of claim 8, wherein the LSECtin-binding moiety further comprises one or more additional CDRL.

11. The composition of claim 5, wherein the CDRH comprises an amino acid sequence of SISSYYSYTS (SEQ ID NO:12) and the additional CDRH comprises an amino acid sequence of VSYSSI (SEQ ID NO:9).

12. The composition of claim 11, wherein the LSECtin-binding moiety further comprises a light chain complementarity determining region (CDRL) having an amino acid sequence of YLAYQSPL (SEQ ID NO:4).

13. The composition of claim 12, wherein the LSECtin-binding moiety further comprises at least a third CDRH having an amino acid sequence of YEEWAYYSSEMAF (SEQ ID NO:18).

14. The composition of claim 13, wherein the LSECtin-binding moiety further comprises one or more additional CDRL.

15. The composition of claim 1, wherein the LSECtin-binding moiety is affinity matured.

16. The composition of claim 1, wherein the antigen is associated with one or more of multiple sclerosis, Celiac disease and/or Type I Diabetes.

17. The compound of claim 1, wherein the antigen comprises a polypeptide comprising (i) portion of SEQ ID NO:26, (ii) a portion of SEQ ID NO:27, (iii) a portion of SEQ ID NO:28, (iv) a portion of SEQ ID NO:26 and a portion of SEQ ID NO:27, (v) a portion of SEQ ID NO:26, a portion of SEQ ID NO:27, and a portion of SEQ ID NO:28, (vi) a portion of SEQ ID NO:23, or (vii) a portion of SEQ ID NO:23 and a portion of SEQ ID NO:80.

18. The compound of claim 1 wherein the antigen comprises a polypeptide comprising a portion of SEQ ID NO:27.

19-20. (canceled)

21. The compound of claim 1, wherein the antigen comprises (i) a polypeptide comprising SEQ ID NO:69 or a polypeptide having at least 90% sequence identity thereto, and SEQ ID NO:70 or a polypeptide having at least 90% sequence identity thereto, (ii) a polypeptide comprising SEQ ID NO:71 or a polypeptide having at least 90% sequence identity thereto and SEQ ID NO:75 or a polypeptide having at least 90% sequence identity thereto, (iii) a polypeptide comprising SEQ ID NO:72 or a polypeptide having at least 90% sequence identity thereto and SEQ ID NO:76 or a polypeptide having at least 90% sequence identity thereto, or (iv) a polypeptide comprising SEQ ID NO:73 or a polypeptide having at least 90% sequence identity thereto and SEQ ID NO:35 or a polypeptide having at least 90% sequence identity thereto.

22.-25. (canceled)

26. The compound of claim 1, wherein the antigen comprises (i) a polypeptide comprising one or more of SEQ ID NO:35, SEQ ID NO:69, SEQ ID NO:70, SEQ ID NO:71, and SEQ ID NO:72, or a polypeptide having at least 90% sequence identity to any of SEQ ID NOs. 35, 69, 70, 71, or 72, or (ii) a polypeptide comprising one or more of SEQ ID NO:73, SEQ ID NO:74, SEQ ID NO:75, SEQ ID NO:76, and SEQ ID NO:35, or a polypeptide having at least 90% sequence identity to any of SEQ ID NOs. 73, 74, 75, 76, or 35.

27. (canceled)

28. The compound of claim 1, wherein the antigen comprises aa polypeptide comprising one or more of SEQ ID NO:35, SEQ ID NO:75, SEQ ID NO:76, SEQ ID NO:71, SEQ ID NO:73, SEQ ID NO:74, and SEQ ID NO:72, or a polypeptide having at least 90% sequence identity to any of SEQ ID NOs. 35, 75, 76, 71, 73, 74, or 72; or (ii) a polypeptide comprising one or more of the amino acids sequences of SEQ ID NO:29, SEQ ID NO:30, SEQ ID NO:31, SEQ ID NO.32, SEQ ID NO:33, SEQ ID NO.34 and SEQ ID NO.35.

29. (canceled)

30. The compound of claim 1, wherein the antigen comprises (i) a polypeptide comprising an amino acid sequence of SEQ ID NO:29, (ii) a polypeptide comprising an amino acid sequence of SEQ ID NO:30 (iii) a polypeptide comprising an amino acid sequence of SEQ ID NO:31, (iv) a polypeptide comprising an amino acid sequence of SEQ ID NO:32, (v) a polypeptide comprising an amino acid sequence of SEQ ID NO:33, (vi) a polypeptide comprising an amino acid sequence of SEQ ID NO:34, or (vii) a polypeptide comprising an amino acid sequence of SEQ ID NO:35.

31.-36. (canceled)

37. The compound of claim 1, wherein the antigen comprises (i) a polypeptide comprising an amino acid sequence of SEQ ID NO:42 or SEQ ID NO:43, or a polypeptide having at least 90% sequence identity to any of SEQ ID NOS. 42 or 43; or (ii) a polypeptide comprising an amino acid sequence of SEQ ID NO:77, SEQ ID NO:78 or SEQ ID NO:79, or a polypeptide having at least 90% sequence identity to any of SEQ ID NOs. 77, 78, or 79.

38-42. (canceled)

43. A method of inducing tolerance to a specific antigen in a subject comprising administering to the subject a compound according to claim 1.

44-48. (canceled)

49. A method for inducing tolerance to a specific antigen in a subject, the method comprising:
administering to the subject a composition comprising:
a binding moiety that binds to human Liver Sinusoidal Endothelial Cell C-Type Lectin (LSECtin) comprising a heavy chain complementarity determining region (CDRH) comprising an amino acid sequence of SISSYY (SEQ ID NO:100),
wherein the LSECtin-binding moiety is an LSECtin-specific antibody or a fragment of an LSECtin-specific antibody; and
an antigen to which tolerance is desired,
wherein the antigen to which tolerance is desired comprises one or more antigens or one or more fragments of said one or more antigens
wherein the antigen to which tolerance is desired is covalently coupled to the LSECtin-binding moiety or joined to the LSECtin-binding moiety via a linker,
wherein a subject exposed to the antigen alone reacts to the antigen alone with an unwanted immune response, and
wherein a subject exposed to the composition has a reduced immune response to a subsequent exposure to the antigen.

50.-89. (canceled)
